(19) United States
(12) Patent Application Publication
(10) Pub. No.: US 2009/0087545 A1
(43) Pub. Date: Apr. 2, 2009

(54) Film Forming Apparatus, Evaporating Jig, and Measurement Method

(76) Inventors: Tadahiro Ohmi, Miyagi (JP); Takaaki Matsuoka, Tokyo (JP); Shozo Nakayama, Aichi (JP); Hironori Ito, Aichi (JP)

Correspondence Address: FOLEY AND LARDNER LLP;SUITE 500, 3000 K STREET NW, WASHINGTON, DC, 20007, US

(21) Appl. No.: 11/992,229

(22) PCT Filed: Sep. 19, 2006
(86) PCT No.: PCT/JP2006/318530

§ 371 Date: Mar. 19, 2008

(30) Foreign Application Priority Data
Sep. 20, 2005 (JP) 2005-272283

(51) Int. Cl. C23C 16/00; B05D 5/12; B05B 7/00; B05B 7/16; G01N 33/00; G06F 19/00

(52) U.S. Cl. 427/66; 118/726; 118/724; 73/23.2; 702/1

(57) ABSTRACT

Provided are an evaporating jig by which a thin film, especially an organic EL film, can be uniformly formed over a long time, and a film forming apparatus including the evaporating jig. The evaporating jig is provided with an evaporating pan, having a bottom plane and side planes arranged to stand from the bottom plane, for defining a material containing space opened inside the side planes; and partitioning plates for partitioning the material containing space into a plurality of partial spaces. The partitioning plates are provided with locking pieces having a height which permits the partial spaces to be continuous on a bottom plane side of the evaporating pan.

### TECHNICAL FIELD

This invention relates to a film forming apparatus for forming a layer of a predetermined material, a jig for use in the film forming apparatus, and a measurement method using the jig and, in particular, relates to a film forming apparatus for forming a layer of a predetermined material by evaporating a raw material of the predetermined material, a jig for use in the film forming apparatus, and a measurement method using the jig.

### BACKGROUND ART

A method of forming a layer of a predetermined material by evaporating a raw material of the predetermined material is widely used in the manufacture of semiconductor devices, flat panel display devices, and other electronic devices. A description will be given hereinbelow using an organic EL display device as one example of those electronic devices. The organic EL display device having a sufficient brightness and a lifetime of several tens of thousands of hours or more uses an organic EL element being a self-light-emitting element and, thus, since peripheral components such as a backlight are small in number, it can be formed thin, and therefore, it is ideal as a flat panel display device.

The organic EL element constituting such an organic EL display device is required in terms of characteristics as a display device such that, while being a large screen, the element lifetime is long, there is no variation in luminous brightness in the screen and element lifetime, and there is no defect such as, typically, a dark spot. In order to satisfy such requirements, the organic EL film forming technique is quite important.

For example, as a film forming apparatus for uniformly forming an organic EL film on a large substrate of about 20 inches, use is made of an apparatus described in Patent Document 1 (Japanese Unexamined Patent Application Publication (JP-A) No. 2004-79904) or the like. The film forming apparatus of Patent Document 1 aims to achieve uniformity in film thickness on a large substrate by optimally arranging, in a tree fashion, a piping structure inside an injector disposed in the apparatus so as to uniformly disperse a raw material gas on the substrate along with a carrier gas.

Recently, an increase in size of 20 inches or more has also been required for this type of organic EL device. However, in order to respond to such a requirement, it is necessary to overcome various drawbacks peculiar to the organic EL device that is poor in light emitting efficiency and short in lifetime. Herein, since various organic EL films, including a light emitting layer, forming the organic EL device are as extremely thin as several tens of nm as compared with films formed in other display devices, a technique of forming a film on a molecular basis is required and, further, it is also quite important to perform the film formation on the molecular basis with high accuracy.

As a film forming apparatus also applicable to the increase in size of 20 inches or more, the present inventors have proposed, in Japanese Patent Application No. 2005-110760 (Prior Application 1), a film forming apparatus for uniformly and quickly forming a film of each of various organic EL raw materials forming an organic EL device.

The proposed film forming apparatus comprises two raw material containers for vaporizing/evaporating the same organic EL raw material, an ejection vessel for ejecting the organic EL raw material onto a substrate, and a piping system (i.e. flow paths) connecting the raw material containers and the ejection vessel to each other. In this case, when supplying the organic EL raw material to the ejection vessel from one of the raw material containers, the piping system including valves and orifices is switched in mode before the start of the film formation, at the time of the film formation, and at the time of stopping the film formation and the temperature of the piping system is controlled. In this structure, during the time other than the film formation, a gas remaining in the piping system is quickly exhausted and a gas is circulated to the other raw material container.

In the film forming apparatus shown in Prior Application 1, it is possible to prevent contamination due to the gas remaining in the piping system and further to quickly perform the state transition before the start of the film formation, at the time of the film formation, and at the time of stopping the film formation. Since the contamination due to the organic EL material remaining in the piping system can be prevented, the film forming apparatus according to Prior Application 1 can significantly improve the brightness and lifetime of an organic EL device.

However, it has been found out that when the structure shown in Prior Application 1 is employed, it is necessary to further improve the use efficiency of the organic EL material forming a light emitting layer or the like of an organic EL device and, for a further increase in size of an organic EL device, it is necessary to further improve the brightness of an organic EL element and to achieve an increase in lifetime of the organic EL element.

Further, in the film forming apparatus shown in Prior Application 1, the evaporated organic EL material is blown into the ejection vessel from one of the raw material containers during the film formation, but is exhausted to the exterior from the one of the raw material containers during the time other than the film formation. In this manner, the organic EL material is effectively used only during the film formation but is not effectively used during the time other than the film formation and, therefore, there has also been found out a drawback that the use efficiency of the using organic EL material is low.

An explanation will be given here of the characteristics and structure of an organic EL device to be achieved. At first, the organic EL device aimed at by this invention is an organic EL device having a long lifetime of 10000 hours or more and a light emitting efficiency of 100 lm/W or more. To briefly explain the structure of the organic EL device according to this invention, it comprises, on a glass substrate, an anode in the form of a transparent conductive film and a cathode made of Li/Ag or the like and provided so as to face the anode, and seven or five organic layers, disposed between the anode and the cathode. Herein, the organic layers are, for example, in the form of an electron injection layer, an electron transport layer, a light emitting layer, a hole transport layer, and a hole injection layer from the cathode side. The light emitting layer comprises, for example, a red light emitting layer, a green light emitting layer, and a blue light emitting layer and, by forming the red light emitting layer, the green light emitting layer, and the blue light emitting layer into a laminated structure in this manner, it is possible to emit white light with high efficiency.

Among the above organic layers, particularly the red light emitting layer, the green light emitting layer, and the blue light emitting layer forming the light emitting layer each have a thickness of about 20 nm and even the electron transport layer and the hole transport layer each have a thickness of about 50 nm. In this manner, the organic layers of the organic EL device are extremely thin as compared with the thicknesses of various films of other semiconductor devices, but, for future, an attempt is made to further reduce the thicknesses of these organic layers. In this case, contamination even on a molecular basis is not allowed for formation of an organic layer. Therefore, in order to deposit/form an extremely thin organic layer with high accuracy, there is required an ultraprecise technology for forming a raw material of an organic layer on a molecular basis.

Patent Document 1: Japanese Unexamined Patent Application Publication (JP-A) No. 2004-79904

### DISCLOSURE OF THE INVENTION

### Problem to be Solved by the Invention

In order to uniformly form an extremely thin organic layer with high accuracy as described above, it is necessary to analyze the characteristics of an organic EL raw material serving as a raw material of the organic layer and further to improve various appliances for use in the film formation, particularly an evaporating jig. That is, an organic EL raw material generally has a characteristic that the thermal conductivity is low and thus the heat is not easily conducted even in the liquid state. Further, although the viscosity of the liquid-state organic EL raw material is not so low, when the temperature thereof is raised for evaporation, the viscosity decreases with the temperature rises so that the thermal convection tends to occur.

On the other hand, in order to evaporate/vaporize the organic EL raw material having such characteristics, an evaporating jig having an evaporating dish is used in Prior Application 1. However, it has been found out that, only with the use of the conventional evaporating dish, there is a limit to the achievable characteristics of an organic EL device and thus the organic EL device having the intended characteristics as described above cannot be obtained.

According to the studies by the present inventors, it has been observed that, in the case of the conventional evaporating dish, the thermal convection of an organic EL raw material occurs in the evaporating dish as the temperature increases and, due to ascending and descending flows caused by the thermal convection, irregularities occur on the liquid surface of the liquid-state raw material and further the liquid surface state changes momently. If the liquid surface changes in this manner, the evaporation rate of the material also changes temporally. This is considered to be one of the reasons why the uniform concentration (evaporation rate) cannot be maintained conventionally. Further, since the liquid-state organic EL raw material is poor in thermal conductivity, the temperature of the liquid-state raw material differs depending on the position in the evaporating dish such that the temperature is high in the vicinity of the heated evaporating dish while it does not tend to rise at its central portion and, as a result of this, the temperature distribution or temperature spots occur. Since the evaporation rate from the evaporating dish changes sensitively in response to the temperature, this is also considered to be one of the reasons why the uniform concentration (evaporation rate) cannot be maintained.

It is an object of this invention to provide a film forming apparatus that can control and laminate, on a molecular basis, films necessary for a display device such as a large organic EL device exceeding 20 inches.

It is another object of this invention to provide a jig suitable for depositing an organic EL raw material.

It is still another object of this invention to provide a measurement method for measuring the concentration of an organic EL raw material in a carrier gas using the above jig.

It is another object of this invention to provide a measurement method that can identify an unknown organic EL raw material.

### Means for Solving the Problem

According to a first aspect of this invention, there is provided a film forming apparatus that evaporates a raw material by evaporation means and supplies the evaporated raw material onto a substrate, thereby forming a film of a predetermined material on the substrate, the film forming apparatus wherein the evaporation means comprises a container having an opening and a bottom surface and a partition member disposed in the container and extending in a direction from the opening toward the bottom surface.

According to a second aspect of this invention, there is provided a film forming apparatus according to the first aspect and wherein the partition member is provided so as to continuously or partially cross the opening and is configured so that a space continues at a bottom portion or a side portion of the partition member.

According to a third aspect of this invention, there is provided a film forming apparatus according to the first aspect and wherein the partition member is configured so as to prevent occurrence of thermal convection and to make a liquid surface uniform when the raw material is in a liquid state and is subjected to an evaporation treatment in the container.

According to a fourth aspect of this invention, there is provided a film forming apparatus according to any one of the first to third aspects and further comprising supply means for a carrier gas that transports the evaporated raw material onto the substrate, wherein a concentration of the raw material in the carrier gas is constant.

According to a fifth aspect of this invention, there is provided an evaporating jig for use in evaporating a filled raw material, the evaporating jig comprising an evaporating dish having a bottom surface and a side surface standing from the bottom surface and defining an opening and a raw material containing space by the bottom surface and the side surface and a partition member received in the raw material containing space and extending in a direction from the opening toward the bottom surface.

According to a sixth aspect of this invention, there is provided an evaporating jig according to the fifth aspect and wherein the partition member is provided so as to continuously or partially cross the opening and is configured so that a space continues at a bottom portion or a side portion of the partition member.

According to a seventh aspect of this invention, there is provided an evaporating jig according to the fifth aspect and wherein the partition member is in contact with the bottom surface at least a portion of a bottom portion of the partition member and has a communication hole near the bottom surface.

According to an eighth aspect of this invention, there is provided an evaporating jig according to any one of the fifth to seventh aspects and wherein the opening has a rectangular shape with a long side and a short side and the raw material containing space has a rectangular parallelepiped shape, wherein the partition member comprises a long-side direction partition piece extending in a long-side direction and a short-side direction partition piece extending in a short-side direction.

According to a ninth aspect of this invention, there is provided a film forming method including causing an organic raw material to be contained in the evaporating jig according to any one of the fifth to eighth aspects and evaporating the organic raw material therein, and transporting the evaporated organic raw material using a carrier gas, thereby depositing a film of the organic raw material on a substrate.

According to a tenth aspect of this invention, there is provided a measurement method that causes an organic raw material to be contained in the evaporating jig according to any one of the fifth to eighth aspects and evaporates the organic raw material therein, and transports the evaporated organic raw material using a carrier gas, thereby measuring a concentration of the evaporated organic raw material in the carrier gas.

According to an eleventh aspect of this invention, there is provided a measurement method according to the tenth aspect and including calculating an activation energy of the organic raw material from the concentration measured by the measurement method according to claim 20.

According to a twelfth aspect of this invention, there is provided a measurement method according to the eleventh aspect and including deriving, from the activation energy obtained by the measurement method according to claim 21, the measured concentration, and a temperature of the raw material, a constant Ko of a formula (1):

V=(Ko/P)×e^{-Ea/kT}

(where Ko is a constant (%·Torr), P is a pressure (Torr), Ea is an activation energy (eV), k is a Boltzmann constant, and T is an absolute temperature)

representing the concentration V (%) of the evaporated organic raw material in the carrier gas.

According to a thirteenth aspect of this invention, there is provided a measurement method according to the twelfth aspect and including predicting the organic raw material from a calculation result of the constant Ko obtained by the measurement method according to claim 22.

According to a fourteenth aspect of this invention, there is provided a measurement method according to any one of the tenth to thirteenth aspects and wherein the organic raw material is a raw material of an organic electroluminescence element.

According to a fifteenth aspect of this invention, there is provided a film forming apparatus that evaporates a raw material used for forming a film of a predetermined material and supplies the raw material evaporated onto a substrate, thereby forming the film of the predetermined material on the substrate, the film forming apparatus wherein evaporation means for evaporating the raw material comprises a heat resistant container having an opening of a predetermined area at one end thereof and adapted to contain therein the raw material in a liquid state and means for dividing the opening of the container into a plurality of partial spaces each having an area smaller than the predetermined area, wherein the dividing means has a portion that continuously or partially crosses the opening and a portion that causes sad partial spaces to communicate with each other at least one of a bottom portion or the opening of the container.

According to a sixteenth aspect of this invention, there is provided an evaporating jig for use in evaporating a filled liquid-state raw material, the evaporating jig comprising an evaporating dish having a bottom surface and a side surface standing from the bottom surface and defining an open raw material containing space inside the side surface and a partition plate dividing the raw material containing space into a plurality of partial spaces, wherein the partition plate is retained in the evaporating dish so that the plurality of partial spaces communicate with each other on a bottom surface side of the evaporating dish.

According to a seventeenth aspect of this invention, there is provided an evaporating jig according to the sixteenth aspect and wherein the evaporating dish defines the raw material containing space having a predetermined length, width, and depth and a rectangular or square opening and the partition plate comprises a partition piece extending in a length direction of the evaporating dish and a partition piece extending in a width direction of the evaporating dish, wherein the partition pieces each have a height smaller than the depth of the raw material containing space.

According to an eighteenth aspect of this invention, there is provided an evaporating jig according to the sixteenth aspect and wherein the side surface has, at its upper portion, a structure that prevents creeping-up of the liquid-state raw material.

According to a nineteenth aspect of this invention, there is provided an evaporating jig according to the sixteenth aspect and wherein the partial spaces are configured so as to each have a polygonal shape as seen from above.

According to a twentieth aspect of this invention, there is provided an evaporating jig for use in evaporating a filled liquid-state raw material, the evaporating jig comprising an evaporating dish having a bottom surface and a side surface standing from the bottom surface and defining an open raw material containing space inside the side surface and a partition plate dividing the raw material containing space into a plurality of partial spaces, wherein the partition plate is configured to be discontinuous in a direction parallel to the bottom surface and is retained in the evaporating dish so that the plurality of partial spaces communicate with each other in the parallel direction.

According to a twenty-first aspect of this invention, there is provided a film forming method including causing a liquid-state raw material to be contained in the evaporating jig according to any one of the sixteenth to twentieth aspects and evaporating the liquid-state raw material therein, and transporting the evaporated raw material using a carrier gas, thereby depositing a film of the raw material on a substrate.

According to a twenty-second aspect of this invention, there is provided a film forming method including causing a liquid-state raw material to be contained in the evaporating jig according to any one of the sixteenth to twentieth aspects and evaporating the liquid-state raw material at a reduced pressure therein, thereby depositing the evaporated raw material on a lower surface of a substrate disposed over the evaporating jig.

According to a twenty-third aspect of this invention, there is provided an evaporating jig according to any one of the sixteenth to twentieth aspects and further comprising means for heating the evaporating dish.

According to a twenty-fourth aspect of this invention, there is provided an evaporating jig according to any one of the sixteenth to twentieth aspects and further comprising means for heating the partition plate.

According to a twenty-fifth aspect of this invention, there is provided an evaporating jig according to the twenty-third or twenty-fourth aspect and wherein the heating means includes a heat pipe.

According to a twenty-sixth aspect of this invention, there is provided an evaporating jig according to any one of the sixteenth to twentieth aspects and further comprising means for supplying a carrier gas to the evaporating dish.

According to a twenty-seventh aspect of this invention, there is provided an evaporating jig according to the twenty-sixth aspect and wherein the carrier gas is supplied through a filter.

### EFFECT OF THE INVENTION

In this invention, there are obtained a film forming apparatus and an evaporating jig that can largely improve the use efficiency of an organic EL raw material and that can accurately control the concentration (evaporation rate) of the organic EL raw material. In the evaporating jig according to this invention, an opening of an evaporating dish (vaporizing dish) is divided by a partition member (partition plate) into a group of small openings (i.e. a group of partitioned regions) each having a size of preferably 5 mm or less, more preferably 3 mm or less, for example, 2.5 mm×2.5 mm, and therefore, the thermal convection hardly occurs in each of the regions partitioned by the partition plate so that rise and depression of the liquid surface due to the thermal convection do not occur in each partitioned region. Further, since the partitioned regions (small openings) communicate with each other, at the bottom of the partition plate, at a height of, for example, 1 to 2 mm with respect to a liquid depth of 5 mm or 0.5 to 1 mm with respect to a liquid depth of 3 mm (in this case, for example, the depth of the container is 5 mm and the height of the partition plate is 4 mm), the liquid surfaces in the partitioned regions become uniform. Therefore, since the uniform liquid surface can be constantly maintained on the whole, the evaporation rate and thus the concentration in a carrier gas can be made temporally uniform. Further, by making the partition plate of a material having good heat conduction and providing heating means such as a heat pipe or a heater also in the partition plate, the temperature of the liquid in the regions (small openings) surrounded by the partition plate can be made constant regardless of place and time, so that temperature spots do not occur. Therefore, using the evaporating jig according to this invention, the evaporation rate of the liquid-state raw material and the concentration can be made temporally uniform.

Further, according to this invention, there is obtained a method of measuring the concentration of an organic raw material in a carrier gas using the above evaporating jig.

### BRIEF DESCRIPTION OF THE DRAWINGS

FIG. 1 is a schematic structural diagram showing a film forming apparatus according to a first embodiment of this invention.

FIG. 2 is a schematic structural diagram showing a film forming apparatus according to a second embodiment of this invention.

FIG. 3 is a diagram for more specifically explaining a piping system, a switcher, and a film forming section of the film forming apparatus shown in FIG. 1 or 2.

FIG. 4 is a diagram showing a part of a film forming apparatus according to a third embodiment of this invention.

FIG. 5 is a diagram showing a film forming section of the film forming apparatus shown in FIG. 4.

FIG. 6 is a timing chart showing switching timings and so on in the film forming apparatus of FIG. 4.

FIG. 7 is a perspective view showing one example of an evaporating dish forming an evaporating jig according to a first example of this invention.

FIG. 8 is a perspective view showing a partition plate forming the evaporating jig according to the first example of this invention.

FIG. 9 is a sectional view showing the relationship between the partition plate and the evaporating dish of the evaporating jig according to the first example of this invention.

FIG. 10 is a plan view for explaining the evaporating jig according to the first example of this invention.

FIG. 11(a) is a diagram for explaining respective portions of an evaporating jig according to a second example of this invention.

FIG. 11(b) is a sectional view taken along A-A′ in FIG. 11(a).

FIG. 11(c) is a sectional view taken along B-B′ in FIG. 11(a).

FIG. 11(d) is a sectional view taken along C-C′ in FIG. 11(a).

FIG. 11(e) is a sectional view taken along D-D′ in FIG. 11(a).

FIG. 11(f) is a diagram for explaining modifications of an upper end portion of a dish member shown in FIG. 11(b) and FIG. 11(e).

FIG. 12 is a sectional view for explaining an evaporating jig according to a third example of this invention.

FIG. 13 is a diagram showing the characteristics when the evaporating jig according to the first, second, or third example of this invention was used, wherein the temperature dependence of the concentration of an organic EL raw material (material H) in a carrier gas is shown in relation to pressure.

FIG. 14 is a diagram showing the characteristics when the evaporating jig according to the first, second, or third example of this invention was used, wherein the pressure dependence of the concentration of an organic EL raw material (material H) in a carrier gas is shown in relation to temperature.

FIG. 15 is a diagram for explaining an evaporating jig according to a fourth example of this invention.

FIG. 16 is a diagram for explaining an application example of the evaporating jig according to this invention.

FIG. 17 is a diagram showing experimental results when the evaporating jig according to this invention was used.

FIG. 18 is a diagram showing the temperature dependence of evaporation behavior of an organic EL raw material (material H) when the evaporating jig according to this invention was used, wherein there is shown the temperature dependence in the state where the pressure was maintained constant.

FIG. 19 is a diagram showing the pressure dependence of evaporation behavior of an organic EL raw material (material H) when the evaporating jig according to this invention was used, wherein there is shown the pressure dependence in the state where the temperature was maintained constant.

### DESCRIPTION OF SYMBOLS

20 organic EL source section
201 organic EL raw material container section
26, 27, 28 film forming section
29 switching section
31 carrier gas piping system
331, 332, 333 piping system
203 evaporating portion
202 partition
261 ejection vessel
262 stage
263 gas dispersion plate
264 filter
30 glass substrate
50 evaporating dish
52 partition plate
521 long-side direction partition piece
522 short-side direction partition piece
54 retaining piece
55 evaporating jig
56 clearance
59 liquid surface at the start of evaporation
62 dish member
64 heat pipe unit
66 cover member
68 heat insulating member
70 heater
72 first partition portion
74 second partition portion
741 heat pipe
76 third partition portion
761 heat pipe
82 upstream filter portion
84 downstream filter portion
86 liquefying container
87 piping

### BEST MODE FOR CARRYING OUT THE INVENTION

Referring to FIG. 1, a film forming apparatus according to a first embodiment of this invention is schematically illustrated. The illustrated film forming apparatus comprises an organic EL source section 20 having a plurality of organic EL sources, first and second film forming sections 26 and 27, and a switching section 29 (switching means) for supplying an evaporated organic EL material from the organic EL source section 20 selectively to the first and second film forming sections 26 and 27. The switching section 29 comprises piping, orifices, mass controllers (flow control systems), valves, and so on. In this connection, the switching section 29 is controlled by a controller (not shown) that controls the valves, the orifices, the flow control systems, and the valves.

Specifically, the illustrated organic EL source section 20 has container sections (hereinafter referred to as raw material container sections) containing organic EL raw materials corresponding to the number of organic EL films to be deposited. For example, in the case of three kinds of organic EL raw materials to be deposited on a glass substrate, the organic EL source section 20 includes three raw material container sections containing the three kinds of organic EL raw materials, respectively. In the case of depositing more kinds of organic EL raw materials, there are provided raw material container sections, containing the organic EL raw materials, corresponding to the number of those raw materials. For example, in the case where organic EL films to be deposited are six layers including an electron transport layer, a red light emitting layer, a green light emitting layer, a blue light emitting layer, an electron blocking layer, and a hole transport layer, six raw material container sections containing raw materials for forming the respective layers are provided in the organic EL source section 20.

Further, in each raw material container section 201 of the organic EL source section 20, there are provided not only an evaporating jig (i.e. an evaporating dish) containing the organic EL raw material for evaporation thereof, but also a heater for heating the organic EL material in the evaporating jig. A carrier gas such as argon, xenon, or krypton is introduced into the evaporating jig of each raw material container section 201 through valves, a flow control system, and a piping system.

Herein, in each raw material container section 201, the carrier gas is introduced and heating is carried out by the heater and, as a result of this, the organic EL material in the evaporating jig is evaporated. Therefore, each raw material container section 201 has a function as evaporation means for evaporating the organic EL material. In the figure, only the single raw material container section 201 is shown in the organic EL source section 20 for simplification of description, but the organic EL source section 20 is further provided with the raw material container sections corresponding to the other organic EL raw materials. In this manner, each raw material container section operates as evaporation means for evaporating the organic EL raw material.

On the other hand, the switching section 29 is provided corresponding to the illustrated raw material container section 201 and, although the same switching sections are provided for the other raw material container sections, respectively, illustration thereof is omitted here for simplification. Carrier gas piping systems 31 (piping, valves, flow control systems, orifices, etc) each for supplying a gas of the same kind as the carrier gas such as argon, xenon, or krypton to the switcher 29 are connected to the switching section 29 and, herein, are provided in one-to-one correspondence with the first and second film forming sections 26 and 27. This carrier gas piping system 31 is carrier gas supply means for supplying the carrier gas to gas ejection means not through the evaporation means.

The illustrated switching section 29 comprises a piping system including therein piping, valves, orifices, flow control systems, and so on and supplies the carrier gas and the evaporated organic EL raw material selectively to the first and second film forming sections 26 and 27.

The first and second film forming sections 26 and 27 have the same structure as each other and, as will be described later, are respectively connected to the switcher 29 through piping systems 331 and 332 having portions with the same piping path length as each other. A description will be given assuming that the illustrated first and second film forming sections 26 and 27 eject and deposit an organic EL raw material evaporated in the illustrated raw material container section 201. However, when depositing a plurality of organic EL raw materials in the first and second film forming sections 26 and 27, respectively, it is necessary to provide a plurality of switchers between a plurality of raw material container sections and the first and second film forming sections 26 and 27 and to provide piping systems (gas flow paths) for connection between the plurality of raw material container sections and the first and second film forming sections 26 and 27 through those switchers.

Each of the first and second film forming sections 26 and 27 comprises an ejection vessel configured to uniformly eject a carrier gas containing the evaporated organic EL raw material onto a glass substrate and a conveyor for conveying the glass substrate on a stage maintained at a constant temperature and operates to eject the carrier gas containing the evaporated organic EL raw material onto the glass substrate from the ejection vessel to thereby deposit an organic EL film thereon. Therefore, the ejection vessel can be called gas ejection means. As is also clear from this, the illustrated film forming apparatus has a plurality of gas ejection means for one evaporation means.

The ejection vessel comprises supply ports arranged such that the organic EL material from the piping system 331, 332 is uniformly dispersed, and a filter for guiding the organic EL material to the glass substrate or the like. The filter may be replaced with a shower plate in the form of a ceramic or metal plate formed with fine holes.

Hereinbelow, the operation of the film forming apparatus shown in FIG. 1 will be described. At first, an organic EL raw material (organic EL molecules) evaporated by heat and a carrier gas is produced from the raw material container section 201. In this state, when the first film forming section 26 is selected by the switching section 29, the organic EL material from the raw material container section 201 is supplied to the first film forming section 26 through the piping system of the switching section 29 and through the piping system 331 in the evaporated state along with the carrier gas. While the organic EL raw material is supplied to the first film forming section 26, the piping system 332 connected to the second film forming section 27 is closed. While film formation is performed in the first film forming section 26, a glass substrate is supplied to an inlet of the second film forming section 27 so that the second film forming section 27 is in a film formation standby state.

When deposition of the organic EL raw material is finished in the first film forming section 26, the organic EL raw material from the raw material container section 201 is supplied to the second film forming section 27 through the piping system 332 due to switching of the piping system by the switcher 29. While film formation is performed in the second film forming section 27, the glass substrate finished with the film formation in the first film forming section 26 is guided by the conveyor to another ejection vessel provided in the first film forming section 26 for forming a film of another organic EL raw material, so that the film formation is carried out using this other organic EL raw material. In other words, different substrates are supplied at different timings to a plurality of gas ejection means corresponding to one evaporation means.

Subsequently, in the same manner as described above, the first and second film forming sections 26 and 27 are controlled to be switched therebetween at the timings determined by the switcher 29 and organic EL raw materials to be deposited are switched in order, so that organic EL films necessary for an organic EL device are deposited on each of the glass substrates moving in parallel.

Herein, the piping system 332 between the switcher 29 and the second film forming section 27 has a length equal to that of the piping system 331 between the switcher 29 and the first film forming section 26 and a piping tree is formed so that film formation is performed under the same conditions. Further, the piping systems 331 and 332 are controlled so that the organic EL raw material is supplied to the first and second film forming sections 26 and 27 at the same flow rate. As a result of this, in the first and second film forming sections 26 and 27, film formation of the same organic EL raw material is selectively carried out under the same conditions.

Therefore, according to this structure, when film formation is finished in one of the film forming sections 26 and 27, film formation can also be performed in the other of the film forming sections 26 and 27 under entirely the same conditions. Further, while a glass substrate finished with film formation is moving in one of the film forming sections 26 and 27, switching is made to the other of the film forming sections 26 and 27 so that the organic EL raw material is supplied to the film forming section after the switching under the same conditions as the one of the film forming sections. Accordingly, the film forming apparatus shown in FIG. 1 can form in order organic EL material films on a plurality of glass substrates in a simultaneous parallel fashion and utilize the organic EL raw material from the raw material container section 201 without waste, thus making it possible to largely improve the use efficiency of the organic EL raw material.

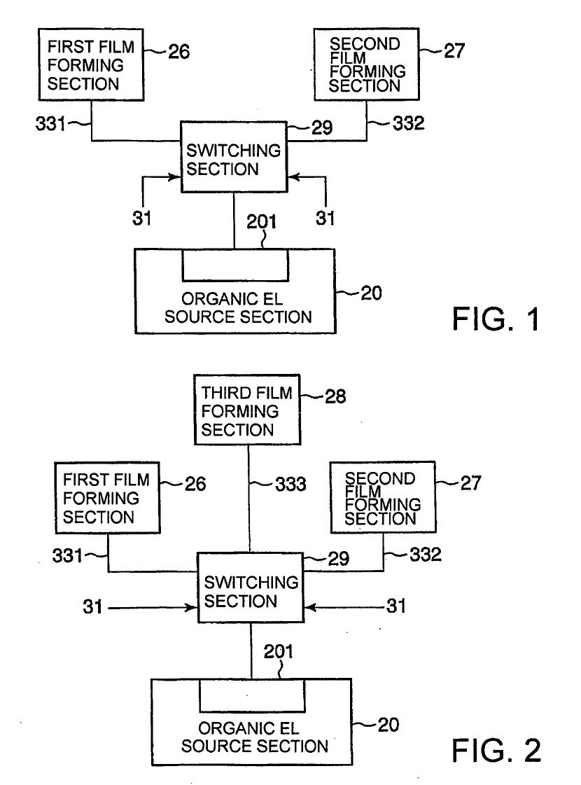

Referring to FIG. 2, there is shown a conceptual diagram of a film forming apparatus according to a second embodiment of this invention. The illustrated example differs from the film forming apparatus of FIG. 1 in that an organic EL raw material from an organic EL source section 20 is individually supplied to three film forming sections, i.e. first to third film forming sections 26 to 28, through a switcher 29, while it is supplied only to the two film forming sections 26 and 27 in the film forming apparatus of FIG. 1. In the illustrated example, the third film forming section is connected to the switcher 29 through a piping system 333 and the piping system 333 is controlled in the same manner as the other piping systems 331 and 332.

At any rate, in the film forming apparatus shown in FIG. 2, an evaporated organic EL raw material from each raw material container section 201 is selectively supplied to the first to third film forming sections 26 to 28 through a switcher 29.

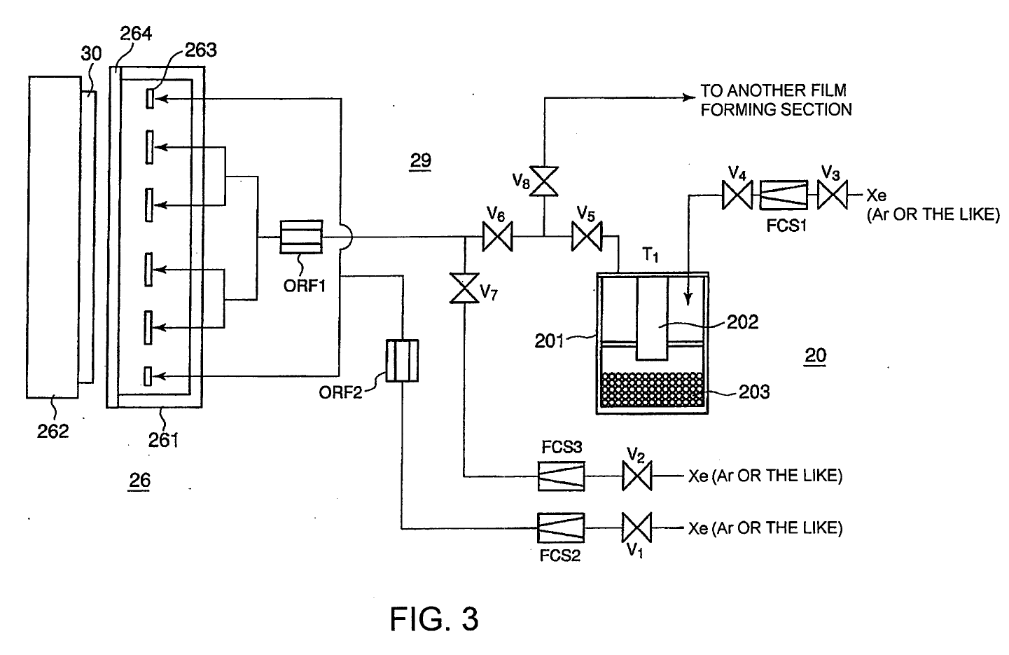

Referring to FIG. 3, there is shown a portion of the film forming apparatus shown in FIG. 1 or 2, wherein the connection relationship among the organic EL source section 20, the switcher 29, and the single film forming section 26 is shown along with a partial structure of the inside of the film forming section 26. The film forming section 26 shown in FIG. 3 comprises an ejection vessel 261 for ejecting a carrier gas containing an organic EL raw material (molecules) in the film forming section 26 and a stage 262 supporting a glass substrate 30. In the state where the glass substrate 30 is mounted thereon, the stage 262 is movable, for example, in a direction perpendicular to the sheet surface of FIG. 3. Further, inside the ejection vessel 261, gas dispersion plates 263 are provided in the number of six in this example and a filter 264 made of metal or ceramic is disposed at a position facing the glass substrate 30. Supply ports are provided corresponding to the gas dispersion plates and both are arranged in a row in the same direction (vertical direction on the sheet surface of FIG. 3). The filter (or a shower plate) has a shape extending in the arranging direction of the supply ports and the gas dispersion plates. The inside of the illustrated film forming section 26 is maintained at a pressure of about 5 to 30 mTorr and the stage 262 is maintained at room temperature.

Herein, the filter 264 is preferably made of a porous ceramic. Generally, when the filter 264 made of the porous ceramic is used, a fluid in the form of a gas or a liquid can be uniformly supplied onto a large-area substrate at a predetermined angle.

On the other hand, the illustrated organic EL source 20 is featured by a single raw material container section 201, wherein the illustrated raw material container section 201 is connected to upstream piping and downstream piping. The upstream piping is piping for introducing a carrier gas into the raw material container section 201 and, as illustrated, includes a flow control system (FCS1) and valves V3 and V4 provided before and after the flow control system FCS1. The downstream piping forms part of the switcher 29.

The raw material container section 201 is divided into an upstream region and a downstream region by a vertically extending partition 202 and an evaporating portion 203 filled with an organic EL raw material is provided under the partition 202. Further, as described before, the raw material container section 201 is provided with a heater (not shown).

In this structure, the carrier gas introduced through the upstream piping is led into the evaporating portion 203 through the upstream region of the raw material container section 201, so that the organic EL raw material (molecules) evaporated in the evaporating portion 203 due to heating by the heater is, along with the carrier gas, led out into the downstream piping through the downstream region of the raw material container section 201.

Like in FIGS. 1 and 2, the switcher 29 is connected to the raw material container section 201. The switcher 29 shown in FIG. 3 comprises a piping system establishing connection between the plurality of film forming sections 26, 27, etc. and the organic EL source section 20 (i.e. the raw material container section 201) and a piping system for supplying a carrier gas to the film forming section 26.

Specifically, a piping system of the switcher 29 establishing connection between the raw material container section 201 and the ejection vessel 261 of the film forming section 26 comprises a first piping system including valves V5 and V6 and an orifice ORF1 and extending to the supply ports corresponding to the four gas dispersion plates 263 provided in the ejection vessel 261 and a second piping system directly leading an externally provided carrier gas source (not shown) of xenon, argon, or the like to the two gas dispersion plates 263 of the ejection vessel 261. The second piping system reaches the supply ports corresponding to the gas dispersion plates 263 of the ejection vessel 261 through a valve V1, a flow control system FCS2, and an orifice ORF2. Further, a third piping system for introducing a gas of the same kind as the carrier gas from the exterior is connected to the first piping system between the orifice ORF1 and the valve V6. This third piping system includes a valve V2, a flow control system FCS3, and a valve V7. Further, a fourth piping system for supplying the evaporated organic EL raw material to another film forming section (e.g. 27 in FIG. 1) is connected to the first piping system between the valves V5 and V6. This fourth piping system includes a valve V8. What is shown as orifice ORF in the figure is a gas pressure adjusting portion having an orifice and a valve for adjusting/controlling a gas pressure. Therefore, the gas pressure adjusting portion is provided between the evaporation means and the ejection vessel, and the gas pressure adjusting portion and the supply ports of the ejection vessel are connected to each other by the piping.

Herein, if, in the first piping system for supplying the carrier gas containing the organic EL raw material (molecules) to the ejection vessel 261, the lengths of the piping between the orifice ORF1 and the supply ports of the ejection vessel 261 are all set equal to each other, it is possible to supply the organic EL raw material (molecular gas) so as to reach the glass substrate 30 uniformly and simultaneously. In this connection, in the illustrated example, the number of the organic EL molecular gas supply ports in the ejection vessel 261 is set to 2ⁿ, and these supply ports and the orifice ORF1 are connected to each other by the piping branched into 2ⁿ paths (n is a natural number). Further, by providing the same piping between the orifice ORF1 and the supply ports of the ejection vessel 261 in each of the plurality of film forming sections, it is possible to uniformly form films of the same organic EL material under the same conditions in the plurality of film forming sections.

Only the carrier gas is supplied to the gas dispersion plates 263 provided at both upper and lower ends in FIG. 3.

Further, the temperature of the first piping system from the raw material container section 201 to the ejection vessel 261 is set higher than the temperature of the raw material container section 201 supplying the organic EL raw material, so as to prevent deposition/adsorption of the organic EL raw material (molecules) on the walls of pipes forming the piping system.

Herein, referring to FIGS. 1 and 3, the operation of the film forming apparatus will be described. At first, the operation of the illustrated film forming apparatus can be classified into operations before the start of film formation, during the film formation, and at the time of stopping the film formation for each of the film forming sections 26 and 27. Herein, a description will be given assuming that the operations before the start of the film formation, during the film formation, and at the time of stopping the film formation are a mode 1, a mode 2, and a mode 3, respectively.

In the mode 1 before the start of the film formation for the film forming section 26, the valves V1, V2, V3, V4, and V7 are in the open state, the valve V6 is in the closed state, and the valves V5 and V8 are in the open state. Accordingly, in the mode 1, the carrier gas is supplied into the ejection vessel 261 through the valve V1, the flow control system FCS1, and the orifice ORF2, while the carrier gas flows into the ejection vessel 261 through the valve V2, the flow control system FCS3, the valve V7, and the orifice ORF1. In this state, the pressure in the ejection vessel 261 and the pressure on the glass substrate 30 are controlled at predetermined pressures. In this case, for example, the pressure in the ejection vessel 261 is controlled at 10 Torr and the pressure on the glass substrate is controlled at 1 mTorr.

Further, in the state of the mode 1, since the valves V3 and V4 are in the open state, the carrier gas to be introduced into the raw material container section 201 that supplies the organic EL molecules is introduced into the raw material container section 201 through the path of the valve V3, the flow control system FCS1, and the valve V4 and, since the valve V6 is in the closed state, the organic EL raw material is not fed to the film forming section 26 but is supplied to the other film forming section (e.g. 27) through the valves V5 and V8 in the open state. Naturally, in a mode before the start of the film formation for the entire film forming apparatus, the valves V5 and V8 are also set to the closed state and, therefore, the organic EL raw material is not fed to either of the film forming sections 26 and 27 from the raw material container section 201 and only the gas of the same kind as the carrier gas is fed thereto through the piping systems provided for both film forming sections, respectively.

In FIG. 3, when the film formation in the first film forming section 26 is started, the state for this film forming section shifts from the mode 1 to the mode 2. In the mode 2 during the film formation, the valves V2, V7, and V8 are set to the closed state, while, the valves V1, V3, V4, V5, and V6 are set to the open state. As a result of this, the carrier gas is fed to the upper and lower supply ports of the ejection vessel 261 through V1, the flow control system FCS2, and the orifice ORF2 and, further, the organic EL molecular gas evaporated in the raw material container section 201 is supplied to the four supply ports of the ejection vessel 261 through the path of V5, V6, and the orifice ORF1 by the carrier gas introduced through the path of the valve V3, the flow control system FCS1, and the valve V4.

In this mode 2, the gas (flow rate f1) of the same kind as the carrier gas that was supplied through the valve V2, the flow control system FCS3, the valve V7, and the orifice ORF1 is stopped. On the other hand, in order to keep constant the pressure in the ejection vessel 261 and the pressure in a chamber, it is preferable that the carrier gas flow rate from the raw material container section 201 serving to supply the organic EL molecules to the ejection vessel 261 be, in principle, set equal to the foregoing flow rate f1. That is, the transport gas flow rate in the path of the valves V5 and V6 and the orifice ORF1 is preferably equal to the flow rate f1 of the gas of the same kind as the carrier gas that was fed in the path of the valve V2, the flow control system FCS3, the valve V7, and the orifice ORF1 in the mode 1.

Next, referring to FIG. 3, the mode 3 at the time of stopping the film formation for the first film forming section 26 will be described. When shifting from the state of the mode 2 to the state of the mode 3, the valve V6 is set to the closed state and the valves V5 and V8 are set to the open state and, simultaneously, the valves V2 and V7 are set to the open state. That is, in the mode 3, the valves V1, V2, V3, V4, V5, V7, and V8 are set to the open state, while, the valve V6 is set to the closed state, so that the organic EL raw material from the raw material container section 201 is supplied to the other film forming section (e.g. 27).

In this manner, in the mode 3, since the valves V5 and V8 are set to the open state, the carrier gas containing the organic EL molecules flows from the raw material container section 201 side to the other film forming section at the flow rate f1 in the mode 2. On the other hand, since the valves V2 and V7 are set to the open state, the gas of the same kind as the carrier gas flows into the ejection vessel 261 of the first film forming section 26 through the orifice ORF1 at the flow rate f1 equal to that in the mode 1. By this gas of the same kind as the carrier gas, the organic EL molecules in the piping from the valve V6, which was in the open state in the mode 2, to the ejection vessel 261 are blown off. Therefore, the expelling of the organic EL molecules is extremely fast in the film forming section 26 at the time of stopping the film formation.

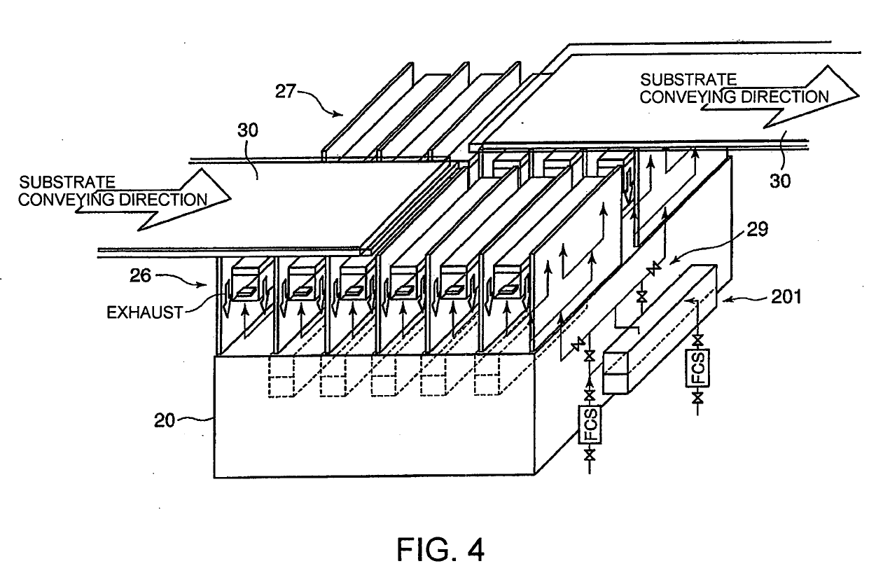

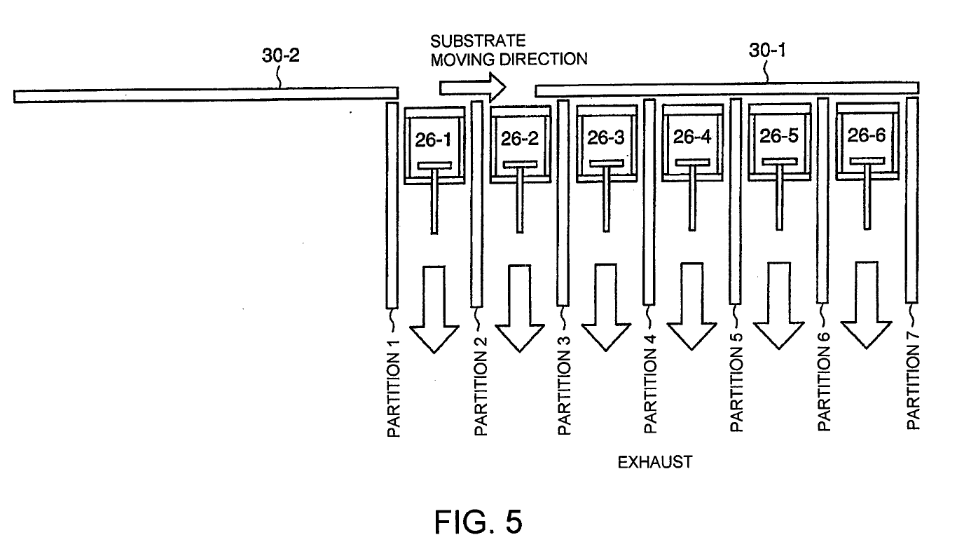

FIG. 4 is a perspective view of a main portion of a film forming system according to another embodiment of this invention. In this embodiment, a film forming section comprises two film forming sections like in the first embodiment, wherein each of the film forming sections 26 and 27 has six ejection vessels. In FIG. 4, the same reference numerals are assigned to portions corresponding to those in the embodiment of FIGS. 1 and 3. The film forming section will be described in detail with reference to FIG. 5. As shown in FIG. 4, in a first film forming section array (chamber CHM1), six ejection vessels each extending to have a length equal to the width of a glass substrate are aligned adjacent to each other so that their length directions are parallel to each other. A glass substrate 30 moves at a predetermined speed over the group of ejection vessels in a direction crossing the above length direction. A second film forming section array (chamber CHM2) is configured in the same manner and another glass substrate 30 is supplied thereover at a timing different from that over the first array. The ejection vessels disposed in the two arrays form pairs and a carrier gas containing a raw material is supplied to each pair at different timings from the same raw material container section. When the carrier gas containing the raw material is selectively supplied to one of the pair of ejection vessels, the glass substrate is present thereover, while, during that time, the carrier gas containing the raw material is not supplied to the other of the pair of ejection vessels and the glass substrate is also not present thereover. Supply/movement of the glass substrates and selection as to which of the pair of ejection vessels the carrier gas containing the raw material is supplied to are cooperatively performed to determine the timing so that the carrier gas containing the raw material is always supplied to either of the pair and the substrate is present thereover.

Referring to FIG. 5, the single film forming section array (chamber) of the film forming system according to the embodiment of FIG. 4 will be described. FIG. 5 shows the single film forming section array for use in manufacturing an organic EL device by forming organic EL films in sequence on a substrate 30 of glass or the like, wherein the films of six layers are formed in sequence on the substrate. In this case, use can be made of a substrate with a size from 730×920 (mm) to 3000×5000 (mm).

The illustrated film forming section array comprises six ejection vessels 26-1 to 26-6 separated by partitions 1 to 7, wherein the ejection vessels eject carrier gases containing organic EL materials onto the glass substrate located above in the order of stacking of the films. These six ejection vessels 26-1 to 26-6 are aligned so that the extending directions of internal filters or shower plates are parallel to each other. Glass substrates 30-1 and 30-2 move, with a fixed interval therebetween, over the six ejection vessels from left to right in the figure and are subjected to formation of organic EL films by the organic EL raw materials ejected upward in the figure from respective ejecting portions of the ejection vessels 26-1 to 26-6. In this event, predetermined distances are maintained between the substrate 30-1, 30-2 and each partition and between the substrate 30-1, 30-2 and each of the ejection vessels 26-1 to 26-6, wherein the distance between the substrate 30-1, 30-2 and each partition is smaller than the distance between the substrate 30-1, 30-2 and each of the ejection vessels 26-1 to 26-6. The gases ejected upward from the respective ejection vessels pass through spaces between the side walls of the ejection vessels and the inner surfaces of the partitions so as to be exhausted downward as shown by arrows. The piping system as shown in FIGS. 3 and 4 is connected to each of the ejection vessels. Therefore, the film forming section array (chamber) shown in FIG. 5 is connected to the non-illustrated other film forming section array (chamber) through the respective piping systems. By controlling the respective piping systems of the plurality of film forming section arrays by respective switchers, it is possible to parallelly process glass substrates in two rows.

In the embodiment of FIG. 5, the glass substrate 30-1, 30-2 has a size of 2,160 mm×2,540 mm and moves in its longitudinal direction. The width of an ejection port of each ejection vessel in the glass substrate moving direction is 50 mm, the length of the ejection port perpendicular thereto is 2,170 mm, the width (thickness) of the side wall of each ejection vessel is 15 mm, the distance between the outer surface of the side wall of each ejection vessel and the inner surface of each of the partitions on both sides thereof is 30 mm, thus the distance between the inner surfaces of the adjacent partitions is 140 mm, the thickness of each partition is 15 mm, and the length of the film forming section array (chamber) in the substrate moving direction is 945 mm. The distance between the upper surface of each ejection vessel and the substrate is 20 mm, the distance between each partition and the substrate is 2 mm, and the temperature of each partition and each ejection vessel is set to 350 to 450° C. The pressure of a film forming atmosphere is 30 mTorr and the ejection speed of the carrier gas containing the raw material ejected from the ejecting portion is 3 m/sec, so that the carrier gas containing the raw material reaches the substrate in 0.1 seconds. The ejection flow rate of the carrier gas containing the raw material from each ejection vessel is 317 cc/min in terms of room temperature and the atmospheric pressure. Assuming that the substrate feed speed is 1.0 cm/sec, the time required for the substrate to pass through one ejection vessel is 264 seconds and the time required for the substrate to pass through six ejection vessels is 341.5 seconds. The use efficiency of the organic EL raw materials reaches 90%.

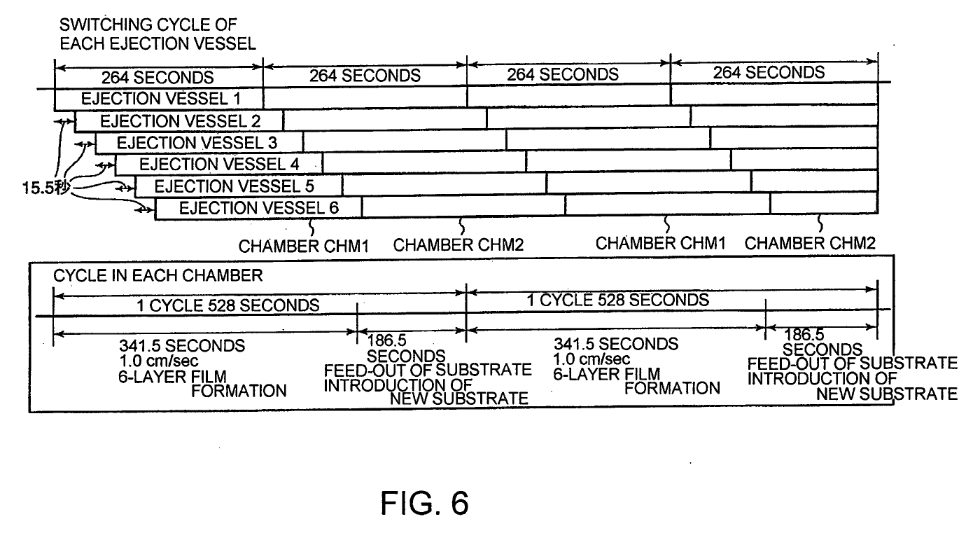

Referring here to FIG. 6, an upper chart is a timing chart showing a switching cycle between the ejection vessels in pairs arranged separately in the two film forming section arrays (chambers), wherein each ejection vessel is subjected to switching of gas supply per 264 seconds. A lower timing chart shows a cycle of the operation in each chamber, wherein, in each chamber, film formation of six layers is achieved in 341.5 seconds and, for 186.5 seconds thereafter, feed-out of a substrate finished with the film formation from the chamber and introduction of a new substrate into the chamber are carried out, so that one cycle is finished in 528 seconds in total. In this one cycle of 528 seconds (8 minutes and 48 seconds), the film formation of 6 layers on the two substrates is completed. The respective ejection vessels in the chamber CHM1, CHM2 are opened with a time lag of 15.5 seconds from each other.

Referring back to FIGS. 3 to 5, all the ejection vessels are made to have completely the same structure, the same piping system described with reference to FIG. 3 is connected to each of them, and the flow rates of the carrier gas to be supplied thereto are also set to the same value. In this case, the temperature of each ejection vessel may be set so as to match the properties of the organic EL molecules. The film forming rate/thickness is preferably controlled by the temperature of each raw material container section. Further, each ejection vessel is preferably made of a stainless steel and the ejecting portion of each ejection vessel is in the form of a stainless filter and is welded to the body. All the inner surfaces of each ejection vessel are preferably coated with a passive film of Al₂O₃ or the like having a low catalytic effect.

Further, in the film forming apparatus according to this invention having the plurality of film forming sections and carrying out the control as described with reference to FIG. 3, the carrier gas flows into the respective film forming sections at completely the same flow rate in either of the modes during the film formation and at the time of stopping the film formation and, therefore, the pressure in the respective ejection vessels forming the respective film forming sections can be maintained constant. This means that cross contamination between the ejection vessels can be prevented.

In the case where the ejection vessels for six layers all have the same size and the flow rates of a carrier gas to be ejected are set to the same value, the concentrations of organic EL raw material molecules in the carrier gas may be set to the same value when the required thicknesses of the respective layers are the same (red light emitting layer, green light emitting layer, blue light emitting layer, electron blocking layer: thickness is 20 to 10 nm for each), while, with respect to the layers with a larger thickness (electron transport layer, hole transport layer: thickness is 50 nm for each), it is necessary to increase the concentration of organic raw material molecules contained in the carrier gas in proportion to the thickness. If this is difficult, it is necessary to take a measure for the layer with the larger thickness to use a plurality of ejection vessels, to increase the opening width of the ejection vessel, to increase the flow rate of the carrier gas, or the like.

Further, as described before, by providing the plurality of film forming sections and temporally switching the modes of these plurality of film forming sections, it is possible to quickly form a plurality of films necessary for an organic EL device and thus to largely improve the throughput and also improve the use efficiency of the organic EL raw materials. For example, in the case of manufacturing an organic EL device by forming organic EL material films of six layers by switching three film forming sections, organic EL devices can be manufactured at intervals of about 6 minutes and, in this case, the use efficiency of the organic EL raw materials can be improved to 82%. As shown in FIGS. 4 to 6, in the case of performing the film formation using the two film forming section arrays, the 6-layer film formation is enabled at intervals of about 8 minutes and the material use efficiency reaches 90%.

Herein, in order to manufacture an organic EL device having the intended characteristics, it is extremely important to keep constant the concentration, in a carrier gas, of an organic EL raw material evaporated from each raw material container section. In other words, if the concentration of the organic EL raw material in the carrier gas changes in a short time, it is impossible to uniformly deposit the organic EL material on a glass substrate or the like on a molecular basis over a long period of time.

However, according to experiments by the present inventors, it has been found out that, in an organic EL manufacturing apparatus currently used by the present inventors, the concentration of each organic EL raw material in a carrier gas rapidly changes in a short time. For example, in the case of Alq3 known as a conventional organic EL raw material, it has been found out that when the concentration of Alq3 in a carrier gas is measured by FT-IR (absorption spectroscopy) in the state where Alq3 is heated to 380° C., Ar as the carrier gas is caused to flow at a flow rate of 10 sccm, and the pressure is maintained at 760 Torr, the concentration reaches a peak (with an overshoot) in a time of about 20 minutes and thereafter rapidly decreases. This also applies to other organic EL raw materials. Such a rapid change in concentration makes it difficult to deposit a uniform organic EL film on a molecular basis over a long period of time. The structure of an evaporating dish in this case is formed by cutting a cylindrical pipe with closed ends into a semicircular shape in section.

As a result of further examining a cause of changing the concentration of the organic EL raw material in the carrier gas, the present inventors have found out that the cause for the change in concentration is due to the evaporating dish used as an organic EL raw material container.

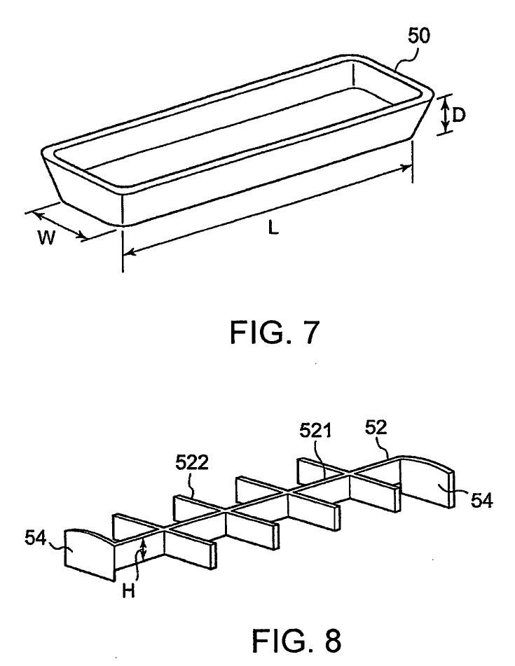

In order to make this clearer, an evaporating dish (i.e. a vaporizing dish) 50 for evaporating an organic EL raw material will be described with reference to FIG. 7. The evaporating dish 50 shown in FIG. 7 is in the form of a rectangular container having a length (L) of 20 mm, a width (W) of 5 mm, and a height (D) of 5 mm. That is, the illustrated evaporating dish 50 has a bottom surface and side walls standing from the bottom surface and extending in the long-side (L) and short-side (W) directions, respectively, so as to define, on the inner side, an open raw material receiving space of a rectangular parallelepiped shape for filling an organic EL raw material therein. In this connection, an opening of the evaporating dish 50 has a rectangular shape with the long and short sides. The evaporating dish 50 used in an experiment is a heat resistant container formed of a heat resistant material, for example, a stainless steel, and serves as evaporation means for evaporating an organic EL raw material.

When the film formation of an organic EL raw material was performed using the evaporating dish 50, although the overshoot was improved and the rapid concentration decrease was also slightly improved, a phenomenon was observed that the concentration of the organic EL raw material in a carrier gas was rapidly lowered so that the constant concentration could not be maintained. This is considered to be caused by the fact that when the illustrated evaporating dish 50 is heated, the organic EL raw material in the evaporating dish 50 is liquefied and, as a result of this, the thermal convection occurs in the liquefied organic EL raw material and changes irregularly and the temperature is also nonuniform, so that the evaporation behavior of the organic EL raw material changes.

Based on this conjecture, the present inventors have assiduously studied a technique that can prevent the thermal convection in the evaporating dish 50 and maintain the temperature therein constant, and have succeeded in developing extremely effective means. Specifically, it has been found out that, by partitioning the organic EL raw material filling space in the evaporating dish 50 into smaller spaces, the influence of thermal convection can be reduced and, as a result, the concentration of the organic EL raw material in the carrier gas can be maintained substantially constant over a long period of time. That is, by providing a partition plate (i.e. dividing means, a partition member) extending in a direction from the opening of the evaporating dish 50 toward the bottom surface thereof so as to divide the filling space into partial spaces, it was possible to reduce the influence of thermal convection, as will be described later.

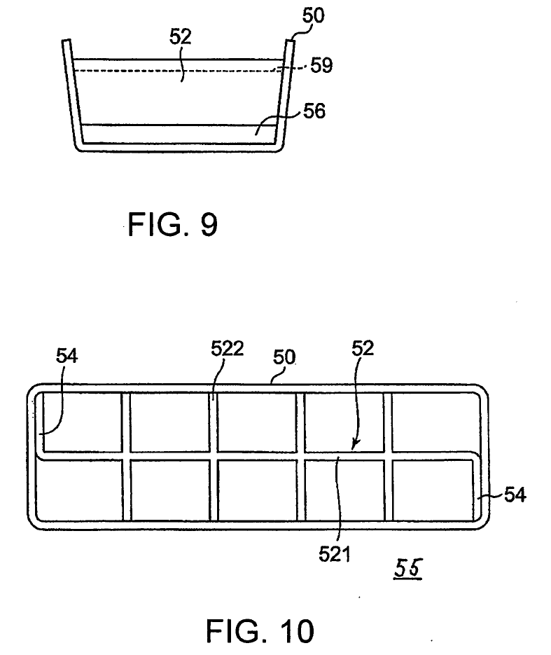

Referring to FIG. 8, a partition plate 52 adapted to be disposed in the evaporating dish 50 is shown as thermal convection preventing means in the evaporating dish 50. The illustrated partition plate 52 is a partition plate (dividing means) for dividing the inner space of the evaporating dish 50 shown in FIG. 7 into 10 small spaces (partial spaces). The illustrated partition plate 52 comprises a long-side direction partition piece 521 having a height (H) of, for example, 3 mm, smaller than a depth (D) of the inner space, four short-side direction partition pieces 522 each having the same height as the long-side direction partition piece, and retaining pieces 54 each having a height (H) of 4 mm and provided at both ends in the long-side direction of the partition plate 52 for contacting/supporting the partition plate 52 on the bottom of the evaporating dish 50. The illustrated two retaining pieces 54 extend from both ends of the long-side direction partition piece 521 in mutually opposite directions (rearward and forward in FIG. 8) with respect to the short-side direction of the evaporating dish 50. Each retaining piece 54 is provided so as to have a greater height than the long-side direction partition piece 521 and the short-side direction partition pieces 522 and to project on the lower side of the long-side direction partition piece 521. Accordingly, the illustrated partition plate 52 is disposed so as to form a clearance 56 between the long-side and short-side direction partition pieces 521 and 522 and the bottom of the evaporating dish 50 as shown in FIG. 9. In other words, there is the clearance 56 between the bottom-side ends of the long-side and short-side direction partition pieces 521 and 522 serving as partition members and the bottom of the evaporating dish 50 and this clearance 56 forms a continuous space along the bottom surface of the evaporating dish 50. As shown in FIG. 9, a liquid surface 59 at the start of evaporation is set lower than the partition plate.

Referring to FIG. 10, there is shown a state where the partition plate 52 shown in FIG. 8 is disposed in the inner space of the evaporating dish 50 so that an evaporating jig 55 is formed. In the illustrated example, the inner space of the evaporating dish 50 is divided into 10 partial spaces (partial regions) by the partition plate 52 and, as is also clear from FIG. 9, the respective partial spaces communicate with each other on the bottom side of the evaporating dish 50. It has been found out that since the illustrated evaporating jig 55 is in the state where the respective partial spaces communicate with each other at the bottom, when an organic EL raw material is liquefied by heating, the liquid surface of the organic EL raw material liquid can be maintained constant and, further, since the inner space is divided into the very small partial spaces, the thermal convection of the organic EL raw material liquid in each partial space can be prevented, so that the concentration of the evaporated organic EL raw material in a carrier gas can be maintained substantially constant over a long period of time.

In the example shown in FIGS. 7 to 10, the description has been given of the case where use is made of the partition plate 52 adapted to form the clearance 56 between itself and the bottom surface of the evaporating dish 50. However, this invention is not limited thereto at all. A partition plate, i.e. a partition wall, may be brought into contact with the bottom surface of the evaporating dish 50. In this case, communication holes may be formed in the partition wall at positions near the bottom surface of the evaporating dish 50 or communication holes may be formed in the bottom of the evaporating dish 50 at positions near the partition plate.

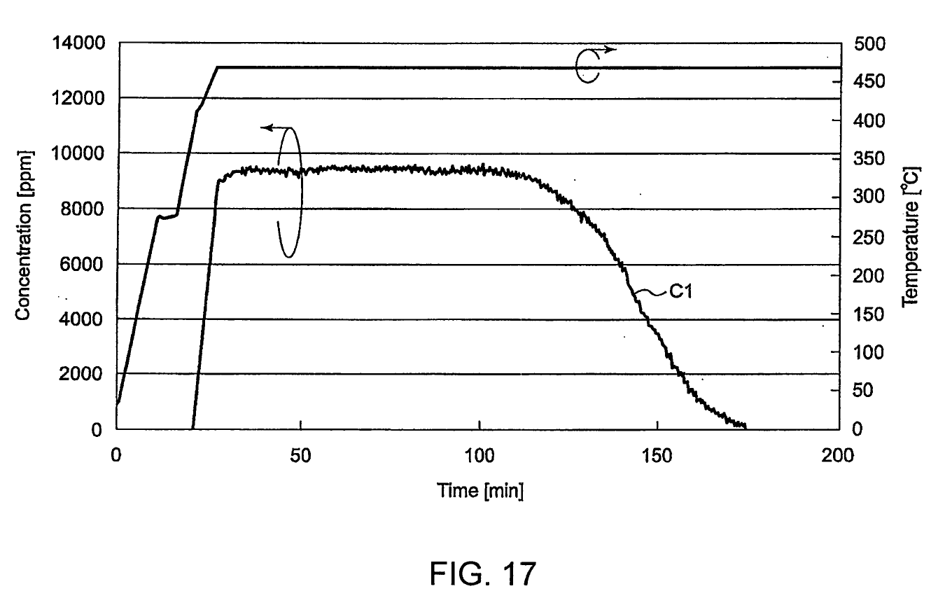

Referring to FIG. 17, there are shown experimental results in the case where use was made of the evaporating dish 50 according to this invention shown in FIGS. 7 to 10. FIG. 17 shows changes C1 in concentration of an organic EL material in a carrier gas when use was made of the evaporating dish 50 of this invention provided with the partition plate. Herein, there is shown the case where Ar was used as a carrier gas and use was made of an organic EL material known as a material H.

In FIG. 17, the curve C1 shows changes in concentration (left scale) of the material H in the carrier gas when 200 mg of the material H was filled in the evaporating dish 50 shown in FIG. 10, maintained at a temperature of 250° C. for 5 minutes, and then heated to 470° C. (right scale). Further, the experiment was conducted by disposing the evaporating dish 50 in an organic EL raw material container maintained at a pressure of 75 Torr and supplying the carrier gas at a flow rate of 10 sccm into the raw material container.

When only the evaporating dish 50 shown in FIG. 7 was used, the highest concentration was obtained at a lapse of 20 minutes after the start of heating, but this concentration started to decrease with a lapse of less than 30 minutes. On the other hand, in the case of C1, the concentration can be maintained at 9000 ppm or more for 100 minutes or more. The fact that the substantially constant concentration can be maintained over a long period of time means that the material H evaporated by the evaporating jig of this invention can be supplied to a film forming section at the constant concentration over a long period of time. Therefore, using the evaporating jig of this invention, an extremely thin film of the material H can be uniformly formed over a long period of time.

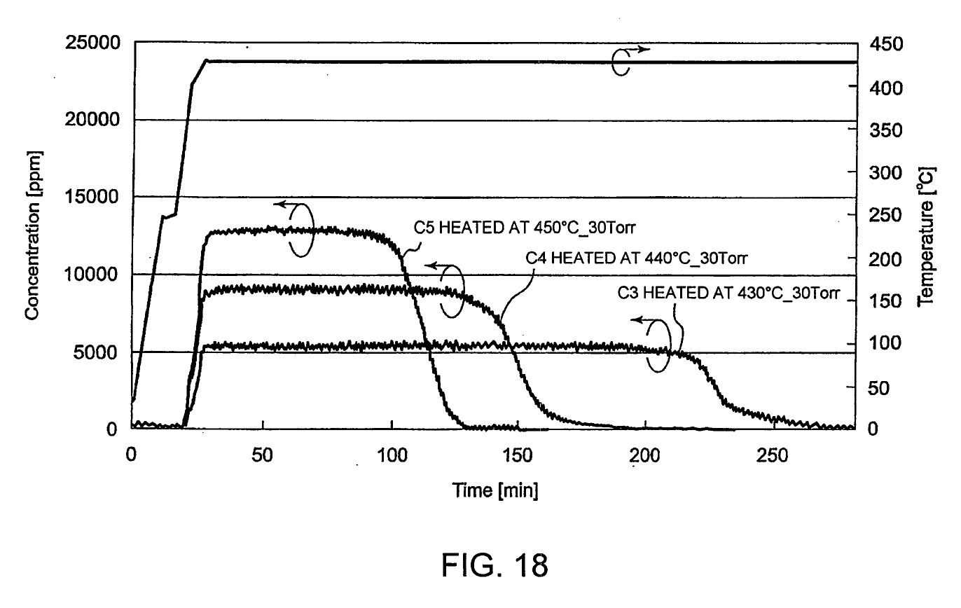

Referring to FIG. 18, there is shown the temperature dependence of evaporation behavior of the organic EL material (herein, the material H), wherein there are shown changes in concentration of the material H when the temperature was changed in the range of 430° C. to 450° C. in the state where the pressure of the evaporating jigs was maintained constant (e.g. at 30 Torr). In this example, there is shown the case where 200 mg of the material H was filled in each of the evaporating jigs according to this invention and the carrier gas was supplied at a flow rate of 10 sccm. A curve C3 shown in FIG. 18 shows a characteristic when the evaporating dish was heated at 430° C. in the state where the pressure was maintained at 30 Torr, wherein the concentration can be maintained substantially constant at about 5000 ppm over a long period of time, i.e. until the organic EL material filled in the evaporating jig is exhausted.

On the other hand, a curve C4 shows concentration changes when heated at 440° C. in the state where the pressure was maintained at 30 Torr. Also in this case, it is possible to maintain a concentration of 9000 ppm for 2 hours or more. Further, a curve C5 shows concentration changes when heated at 450° C. in the state where the pressure was maintained at 30 Torr, wherein a concentration of 13000 ppm can be achieved and this concentration can be maintained until substantially all the filled organic EL material is evaporated from the evaporating jig.

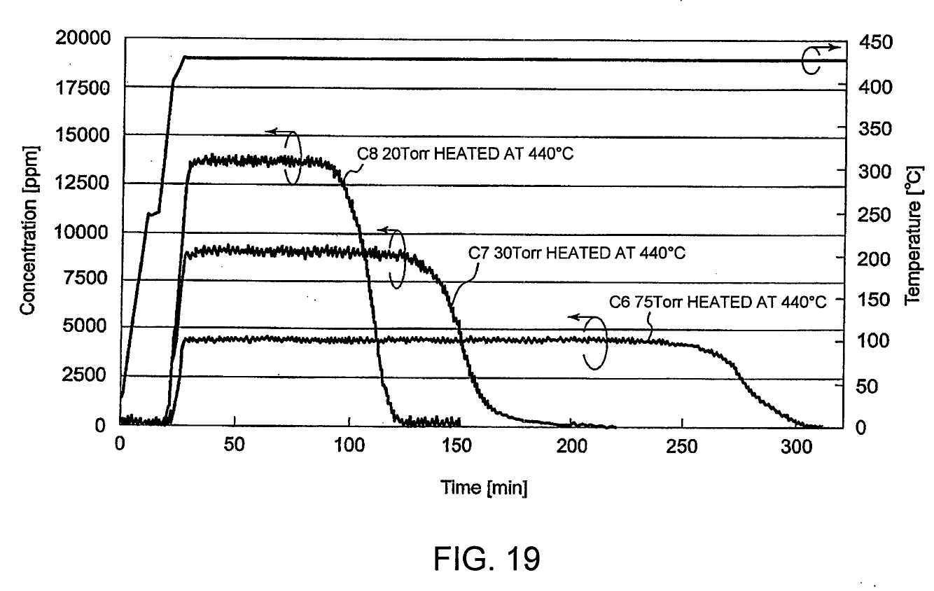

Referring to FIG. 19, there are shown the pressure dependence characteristics of evaporation behavior of the material H being the organic EL material. In this example, evaporating jigs according to this invention are maintained at a temperature of 440° C. and Ar is supplied as a carrier gas to the evaporating jigs at a flow rate of 10 sccm. Like in FIGS. 17 and 18, 200 mg of the material H is filled in each evaporating jig. Curves C6, C7, and C8 show evaporation characteristics of the material H in the states where the evaporating jigs were maintained at 75 Torr, 30 Torr, and 20 Torr, respectively. As is also clear from these curves C6 to C8, the concentration of the material H in the carrier gas increases as the pressure decreases and, in any of the cases, the concentration of the material H in the carrier gas can be maintained substantially constant.

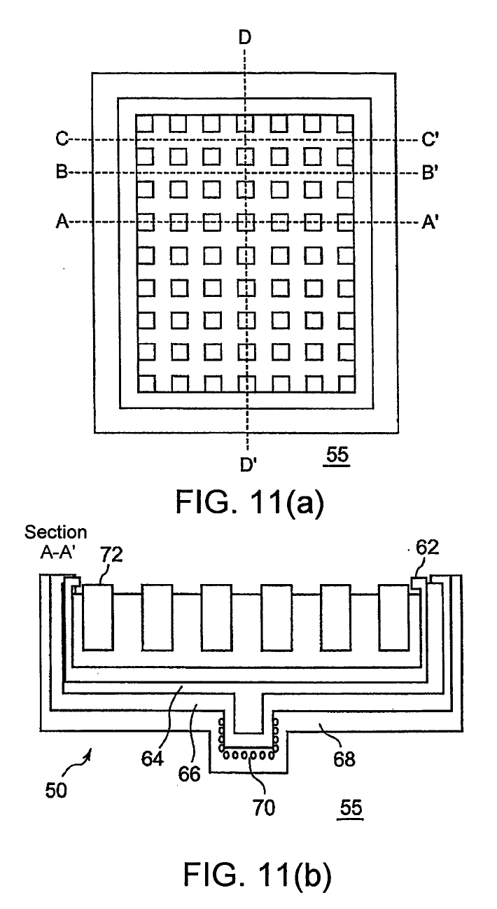

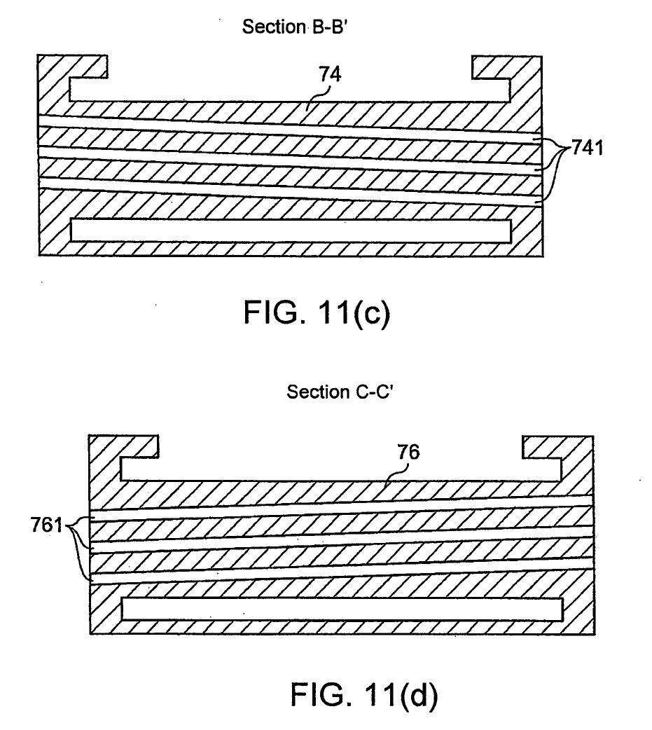

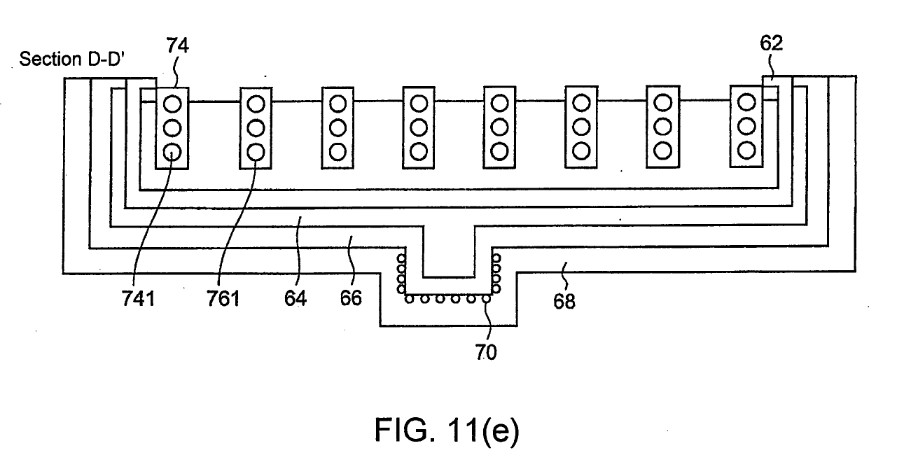

Referring to FIGS. 11(a), (b), (c), (d), (e), and (f), an evaporating jig 55 according to a more specific example (second example) of this invention will be described. FIG. 11(a) shows a plan view of the evaporating jig 55 according to the second example of this invention, wherein, as is clear from the figure, an evaporating dish 50 is divided into 7 partial regions in the lateral direction and 9 partial regions in the longitudinal direction, i.e. 63 partial regions in total, by a partition plate forming a partition member.

Accordingly, hereinbelow, a description will be made given that the lateral direction in FIG. 11(a) is a short-side direction of the evaporating jig 55 and the longitudinal direction in FIG. 11(a) is a long-side direction of the evaporating jig 55. Herein, referring to FIG. 11(b) in the form of a sectional view taken along A-A′ in FIG. 11(a), the evaporating dish 50 of the evaporating jig 55 comprises a dish member 62 for containing an organic EL raw material therein, a heat pipe unit 64 provided so as to cover the outer periphery of the dish member 62, a cover member 66 covering the heat pipe unit 64, and a heat insulating member 68 provided on the outer periphery of the cover member 66. Herein, the dish member 62 is formed of a material having good heat conduction, excellent mechanical strength, and a small thermal expansion coefficient, for example, Cu—W, Al—Mg—Zn, or the like, and has inner surfaces coated with Y₂O₃, Al₂O₃, carbon, or the like. The cover member 66 is formed of a stainless steel or the like having a high heat-insulating effect. The illustrated evaporating dish 50 is composed of the dish member 62, the heat pipe unit 64, the cover member 66, and the heat insulating member 68. Further, as illustrated, a heater 70 is provided at the bottom of the evaporating dish 50.

On the line A-A′ crossing the partial regions, first partition portions 72 each having a height small enough not to reach the bottom of the dish member 52 are provided for defining the seven partial regions. No heat pipe is provided in the illustrated first partition portions 72.

Referring to FIGS. 11(c) and (d), there are shown second and third partition portions 74 and 76 disposed along lines B-B′ and C-C′ in FIG. 11(a), respectively. Heat pipes 741 are embedded in the second partition portion 74 so as to be inclined downward in the right direction in FIG. 11(c).

On the other hand, heat pipes 761 are embedded also in the third partition portion 76 shown in FIG. 11(d) so as to be inclined downward in the left direction as shown in FIG. 11(d).

As shown in FIGS. 11(c) and (d), the heat pipes 741 and 761 embedded in the second and third partition portions 74 and 76 are inclined so as to allow a liquid to flow therein. The partition portions 74 and 76 provided therein with the right-downward and left-downward heat pipes 741 and 761 are alternately arranged in the dish member 52 in FIG. 11(a). Therefore, the partition plate shown in FIG. 11 is formed by joining the first to third partition portions 72, 74, and 76 together.

The inner surfaces of the respective heat pipes 741 and 761 are coated with Y₂O₃ or the like so as to prevent occurrence of a solvent decomposition effect by a catalyst.

Referring to FIG. 11(e), there is shown a section of the partition plate taken along line D-D′ in FIG. 11(a), wherein, as is also clear from the figure, it is seen that the second and third partition portions 74 and 76 are alternately disposed and the right-downward and left-downward heat pipes 741 and 761 are embedded in these second and third partition portions 74 and 76, respectively.

Further, the second and third partition portions 74 and 76 are disposed in the dish member 62 so as to have a predetermined distance from the bottom of the dish member 62, i.e. so as not to be in contact with the bottom of the dish member 62. Therefore, with this structure, since the respective partial regions defined by the first to third partition portions 72, 74, and 76 communicate with each other at the bottom of the dish member 62, when an organic EL material is liquefied, the surface of the liquefied organic EL material is maintained constant in the respective partial regions.

The partition plate shown in FIGS. 11(a) to (e) can be easily produced by interposing, as spacers, the first partition portions 72 with no heat pipe embedded, between the second and third partition portions 74 and 76.

These first to third partition portions are formed of a material having good heat conduction, excellent mechanical strength, and a small thermal expansion coefficient, for example, Cu—W, Al—Mg—Zn, or the like, and the outer surfaces thereof are coated with Y₂O₃, Al₂O₃, carbon, or the like.

Herein, the partition plate shown in FIG. 11 forms the partial regions each having a size of 2 to 5 mm×2 to 5 mm with a thickness of 0.5 to 2 mm.

In a specific example produced separately, a dish member has a size with a long side of 123 mm, a short side of 72 mm, and a depth of 30 mm, each of second and third partition portions (corresponding to 74 and 76 in FIG. 11) has a plate thickness of 3 mm and is embedded with heat pipes each having a diameter of 2 mm, while, first partition portions (corresponding to 72 in FIG. 11) each have a plate thickness of 2 mm. Partial regions surrounded by the first to third partition portions 72, 74, and 76 each have a rectangular shape of 5 mm×3 mm (the distance between the partition portions with the heat pipes embedded is set to 5 mm) and the 200 partial regions in total are arranged.

Referring to FIG. 11(f), there are shown modifications of an upper end portion of the dish member 62 shown in FIGS. 11(b) and (e). That is, when an organic EL raw material was liquefied, a phenomenon was observed that the liquefied raw material crept up the inner walls of the evaporating dish 50 to be leaked out to the outside. A case was observed where, in order to prevent such creeping-up of the liquid-state raw material, only bending the upper end portion of the dish member 62 to the inner side of the evaporating dish 50 as shown in FIGS. 11(b) and (e) was insufficient depending on an organic EL raw material. In the case of an evaporating dish 50 that vaporizes/evaporates such an organic EL raw material, it is possible to prevent creeping-up of the liquid-state raw material by not only bending an upper end portion of a dish member 62 to the inner side of the evaporating dish 50, but also, as shown in FIG. 11(f), inclining a bent portion of the upper end portion at an acute angle with respect to the liquid surface or folding the upper end portion to the inner side of the evaporating dish 50 into a hook shape.

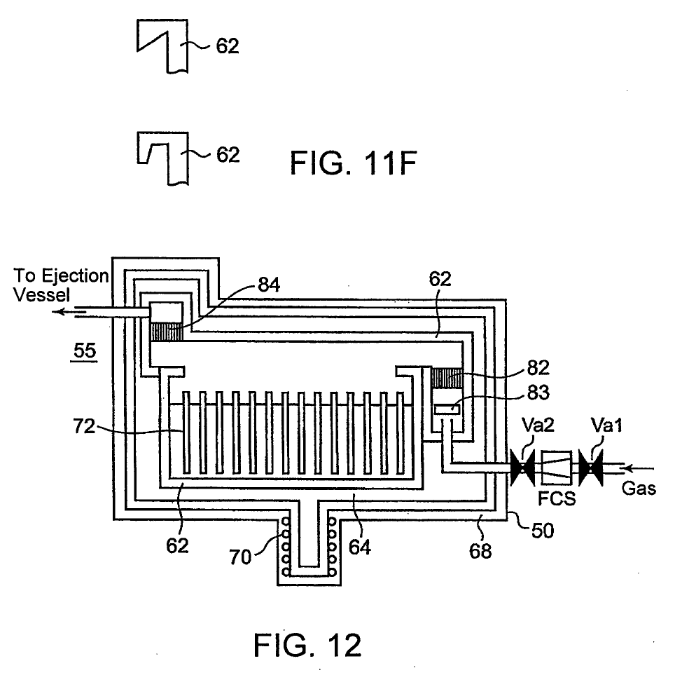

Referring to FIG. 12, an evaporating jig (also referred to as an evaporating container) 55 according to another example of this invention will be described. The illustrated evaporating jig 55 has a structure suitable for achieving uniform flow of a carrier gas. Specifically, as described in relation to FIG. 11(a), the illustrated evaporating jig 55 comprises an evaporating dish 50 having a short-side direction and a long-side direction and partition portions 72 disposed in the evaporating dish 50. As is also clear from this, in FIG. 12, like in FIG. 11(b), there is shown a short-side direction section of the evaporating dish 50. The partition portions 72 are configured so as not to be in contact with the bottom of the evaporating dish 50 and, as a result of this, partial regions defined by the partition portions 72 communicate with each other at the bottom of the evaporating dish 50. The illustrated evaporating dish 50 has a dish member 62, a heat pipe unit 64, a cover member 66, a heat insulating member 68, and a heater 70, which is also the same as the evaporating dish shown in FIG. 11(b).

Further, the illustrated evaporating jig 55 is attached with an upstream filter portion 82 provided on the short-axis direction upstream side and a downstream filter portion 84 provided on the short-axis direction downstream side. The upstream filter portion 82 and the downstream filter portion 84 are each provided with a slit filter formed of Al₂O₃, Y₂O₃, or the like. The upstream filter portion 82 is connected to a carrier gas supply source through a valve Va1, a flow control system (FCSa), and a valve Va2, while, the downstream filter portion 84 is connected to an ejection vessel.

In the illustrated evaporating jig (evaporating container) 55, a carrier gas is caused to flow in its short-side direction. As is also clear from this, the upstream filter portion 82 and the downstream filter portion 84 operate as supply means for the carrier gas that transports a raw material vaporized/evaporated in the evaporating jig 55 onto a non-illustrated substrate. In this case, the gas pressure in the evaporating container is maintained at about 20 Torr and the gas discharged to the ejection vessel from the downstream filter portion 84 is maintained at a pressure of 10 Torr or more and supplied at a gas flow rate of 10 cc/mm. Therefore, the pressure drops in the upstream filter portion 82 and the downstream filter portion 84 are preferably adjusted to about 700 Torr and about 10 Torr or less (preferably 5 Torr), respectively.

When the evaporating dish 50 has a width of 10 cm or 20 cm in the long-side direction, assuming that a gas is caused to flow at 10 cc/min per width of 5 mm, the total gas flow rate becomes 200 cc/min or 400 cc/min. A baffle plate 83 is provided inside a gas supply port so as to ensure uniform flow of the gas.

When the evaporating jigs 55 shown in FIGS. 7 to 10, 11, and 12 were applied to the film forming apparatuses shown in FIGS. 1 to 4, it was possible to extremely accurately and stably form an organic film having a predetermined thickness. For example, when a material H or a material C used as an organic EL raw material was filled in the evaporating jig 55, it was possible to stably vaporize/evaporate the organic EL material at a predetermined concentration over a long period of time. In this case, it was confirmed that the organic EL material could be vaporized/evaporated at a predetermined concentration by maintaining constant either one of the temperature and the pressure in an evaporating container where the evaporating jig 55 was disposed.

As described with reference to FIG. 18, in the case of, for example, the material H, when the evaporating jig 55 shown in FIGS. 7 to 10, 11, or 12 is heated while maintained at a constant pressure, the concentration determined by a temperature can be maintained substantially constant over a long period of time. Further, as described with reference to FIG. 19, when the temperature is maintained constant, the evaporation characteristics of the material H are such that the concentration of the material H in a carrier gas increases as the pressure decreases and the concentration of the material H in the carrier gas can be maintained substantially constant at any of the pressures.

Likewise, similar relationships were obtained with respect to other materials (e.g. a material C) known as organic EL raw materials.

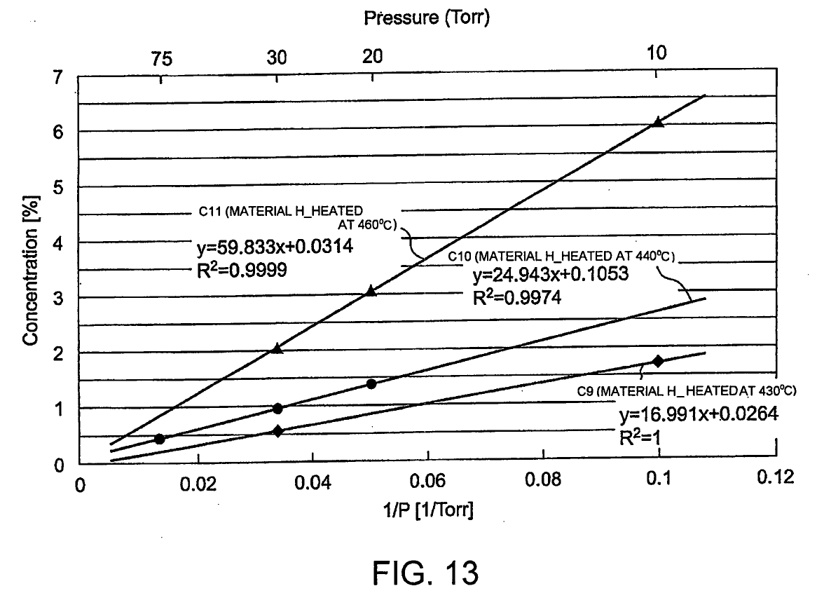

Referring to FIG. 13, there is shown the relationship between concentration and pressure in the state where the temperature of each evaporating jig 55 shown in FIGS. 7 to 10, 11, or 12 was maintained constant. In FIG. 13, for reference, the upper scale is graduated in Torr and the lower scale is graduated in 1/P (1/Torr). In FIG. 13, a characteristic C9 represents the relationship between the pressure and the concentration of the material H in the carrier gas when the material H was heated at 430° C. and, likewise, characteristics C10 and C11 represent the characteristics when the material H was heated at 440° C. and 460° C., respectively. Herein, given that the concentration on the axis of ordinates is y and (1/P) of the lower scale of the axis of abscissas is x in FIG. 13, the characteristic C9 can be approximated by a straight line of y=16.991x−0.0264 and, likewise, the characteristics C10 and C11 can be approximated by straight lines of y=24.943x+0.1053 and y=59.833x+0.0314, respectively.

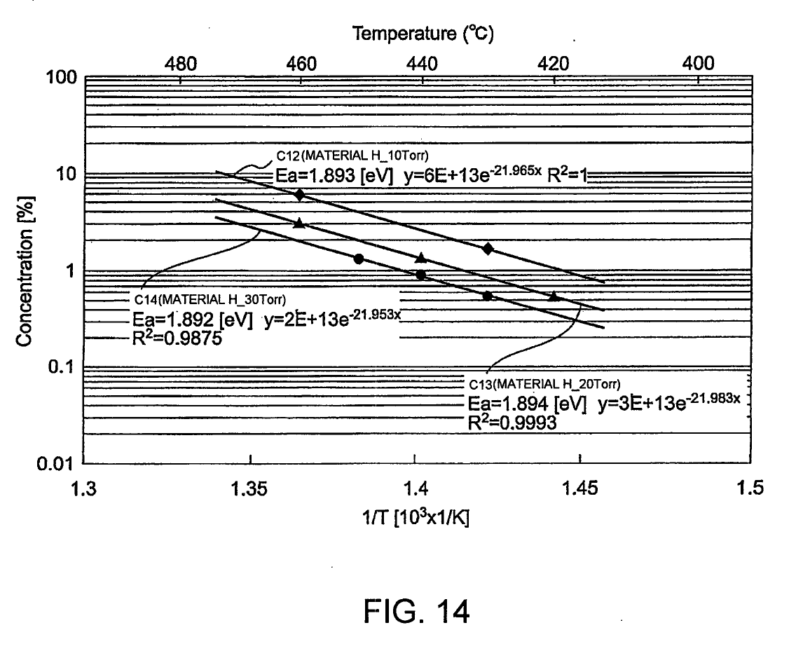

Herein, when the logarithms of the concentrations y in FIG. 13 are plotted against an inverse number of an absolute temperature (1/T)(10³×1/K), characteristics C12, C13, and C14 in FIG. 14 are obtained. Herein, the characteristic C12 shows the plotted results at 10 Torr and, likewise, the characteristics C13 and C14 show the plotted results at 20 Torr and 30 Torr, respectively. Further, the characteristics C12, C13, and C14 can be approximated by y=6E+13e^{-21.965x}, y=3E+13e^{-21.983x}, and y=2E+13e^{-21.953x}, respectively. Herein, x is a value of 1/T expressed by the absolute temperature.

From the above formulas and the characteristics C12 to C14, the slopes of the characteristics C12 to C14 represent activation energies Ea in constant pressure states of 10 Torr, 20 Torr, and 30 Torr, respectively, and values thereof are 1.893 eV, 1.894 eV, and 1.892 eV, respectively.

On the other hand, the evaporation rate of the material H, i.e. the concentration of the material H, can be represented by the following formula (I).

V(%)=(Ko/P)×e^{-Ea/kT} (1)

where Ko is a constant (%-Torr), P is a pressure (Torr), k is a Boltzmann constant (=8.617×10⁻⁵ eV/K), and Ea is an activation energy (eV). Since the material H concentrations given by the formula (I) should be equal to the formulas derived from FIG. 13, i.e. y=6E+13e^{-21.965x}, y=3E+13e^{-21.983x}, and y=2E+13e^{-21.953x}, the constant Ko can be derived from the formula (I) and the formulas obtained from FIG. 14 by giving the temperatures and the material H concentrations.

Tables 1, 2, and 3 show material H concentrations in 10 cc/min at pressures of 10, 20, and 30 Torr, respectively, and values of Ko.

TABLE 1
Material H Concentration in 10 cc/min at a pressure of 10 Torr

| Temperature | Material H Concentration | K (Constant) Value |
|---|---|---|
| 430° C. | 1.68% | 5.903 × 10¹⁴ (% · Torr) |
| 460° C. | 6.01% | 5.908 × 10¹⁴ (% · Torr) |
| 000° C. | 0.00% | 0.000 × 10¹⁴ (% · Torr) |

TABLE 2
Material H Concentration in 10 cc/min at a pressure of 20 Torr

| Temperature | Material H Concentration | K (Constant) Value |
|---|---|---|
| 420° C. | 0.54% | 5.968 × 10¹⁴ (% · Torr) |
| 440° C. | 1.37% | 6.220 × 10¹⁴ (% · Torr) |
| 460° C. | 3.05% | 5.992 × 10¹⁴ (% · Torr) |

TABLE 3
Material H Concentration in 10 cc/min at a pressure of 30 Torr

| Temperature | Material H Concentration | K (Constant) Value |
|---|---|---|
| 430° C. | 0.54% | 5.708 × 10¹⁴ (% · Torr) |
| 440° C. | 0.91% | 6.211 × 10¹⁴ (% · Torr) |
| 450° C. | 1.28% | 5.710 × 10¹⁴ (% · Torr) |

The constant Ko of the material H is derived in Tables 1 to 3. When a material is unknown, if a measured value of the concentration at a particular temperature is obtained and further an activation energy Ea is obtained from the temperature dependence of the organic EL raw material concentration like that shown in FIG. 14, a value of the constant Ko is determined and, by comparing this value with Tables 1 to 3, the unknown material can be identified as the material H.

Likewise, the same evaluation as that of the material H was also carried out for an organic EL raw material known as a material C. As a result of this, the results similar to those on the material H were obtained.

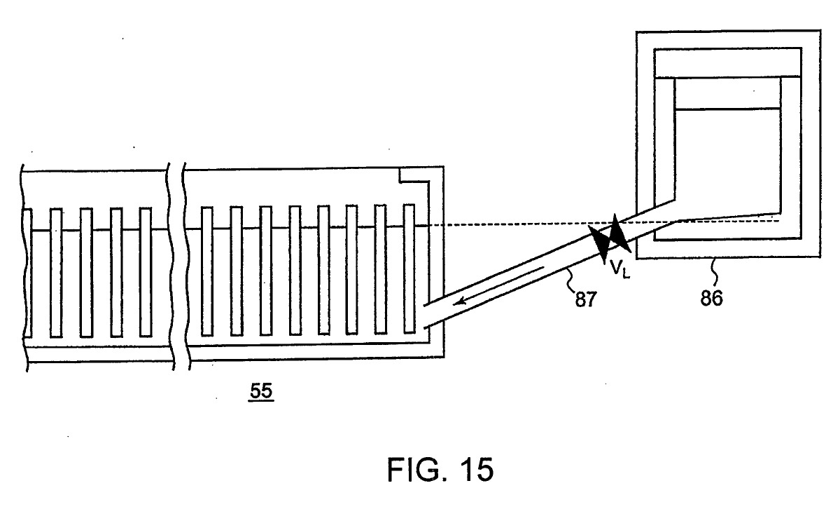

Referring to FIG. 15, there is shown an evaporating jig 55 according to a fourth example of this invention. In the description of the evaporating jigs 55 shown in FIGS. 10 and 11, it has been described that the solid organic EL raw material is filled therein and then heated to be melted into a liquid state. However, when filling the solid organic EL raw material into the evaporating jig 55, the organic EL raw material is inevitably exposed to air and, therefore, it is considered that the quality of the solid organic EL raw material may be degraded due to oxidation or the like. In consideration of this, an evaporating container section shown in FIG. 15 has a structure in which a liquefying container 86 is connected to an evaporating jig 55. The illustrated liquefying container 86 has an inclined bottom surface and is connected to the evaporating jig 55 through piping 87 having a valve VL. Further, the liquefying container 86 is provided with a heater for heating a solid organic EL raw material filled therein.

Into the liquefying container 86 of the illustrated evaporating container section, at first, a solid organic EL raw material is filled in a clean N₂ atmosphere (or a clean dry air atmosphere). Subsequently, in the state where clean Ar or N₂ is caused to flow at the atmospheric pressure, the solid organic EL raw material is baked in the liquefying container 86, thereby removing contaminants adhering to the solid organic EL raw material. Further, the liquefying container 86 is slowly heated at the atmospheric pressure, so that the organic EL raw material is liquefied, for example, at 250° C. When the organic EL raw material has been liquefied, the valve VL is opened, so that the liquid-state organic EL raw material is, due to its own weight, filled into the evaporating jig 55 from the liquefying container 86 through the piping 87 attached to the liquefying container 86 near its bottom. In the case where the viscosity of the liquefied organic EL raw material is large enough to prevent the smooth flow, it may be considered to pressurize the liquid-state organic EL raw material using a gas so as to expel it into the evaporating jig 55 from the liquefying container 86.

With this structure, it is possible to fill the liquid-state organic EL raw material into the evaporating jig 55 without bringing the liquid-state organic EL raw material into contact with the air.

It is also possible to employ a structure in which a partition plate is provided in the liquefying container 86 like in the evaporating container 55, thereby uniformly heating the organic EL raw material.

In the foregoing examples, the description has been given of the cases where the evaporating jigs 55 are used in the film forming apparatuses shown in FIGS. 1 to 6. However, the evaporating jig according to this invention is not limited thereto, but is also applicable to film forming apparatuses of other types. For example, also in the film forming apparatus described in Prior Application 1 or in a film forming apparatus and method in which a plurality of evaporation means are provided for one film forming section, the evaporating jig according to this invention is likewise applicable as the evaporation means. Further, the evaporating jig according to this invention can be used for supplying an evaporated raw material contained in a carrier gas not only to those apparatuses, as described in FIGS. 1 to 6 and Prior Application 1, each having an ejection structure as a film forming section for ejecting a carrier gas containing an evaporated raw material toward a substrate, but also to other film forming mechanisms such as a microwave-excited plasma apparatus.

In addition thereto, the evaporating jig according to this invention can also be used as a deposition jig in a deposition apparatus.

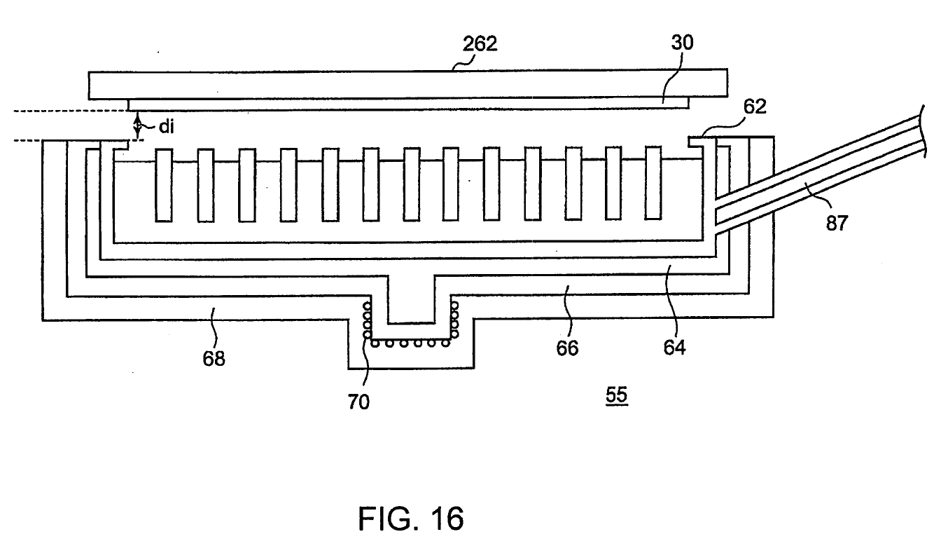

Referring to FIG. 16, there is shown an example where an evaporating jig 55 of this invention is applied to a deposition apparatus (i.e. a vacuum deposition apparatus). The illustrated deposition apparatus has a deposition jig 55 of this invention and a substrate 30 (e.g. a glass substrate) placed on a stage 262 in a face-down manner so as to face the deposition jig 55. Like the evaporating jig 55 shown in FIG. 11, the illustrated evaporating jig 55 comprises a dish member 62, a heat pipe unit 64, a cover member 66 covering the heat pipe unit 64, and a heat insulating member 68 provided on the outer periphery of the cover member 66. Further, a distance di between the substrate 30 and an upper end portion of the evaporating jig 55 is set to 3 to 20 cm (e.g. 5 cm).

Herein, at first, a description will be given of the case where an organic EL raw material having a mean free path of about 10 m at 10⁻⁴ Torr is deposited on the substrate placed on the stage 262 in the face-down state (the state where a film-forming surface faces downward). In this case, the organic EL raw material is filled into the evaporating jig 55 from a non-illustrated liquefying container through piping 87. On the other hand, the substrate 30 is placed on the stage 262 in the state where the temperature is maintained at 100° C. In this state, when the evaporating jig 55 is evacuated to about 10⁻⁴ Torr and heated to about 250° C., since the mean free path of the organic EL raw material is about 10 m, the organic EL raw material is evaporated at a temperature which is lower than that in the case of using a carrier gas (about 350° C.) by 100° C. or more. As a result of this, the formation of an organic EL film on the substrate 30 is completed in a short time of about 30 to 60 seconds. Since the evaporation rate is in inverse proportion to the pressure, the film formation can be stopped by increasing the pressure of an atmosphere of the evaporating jig 55 to 1 to 10 Torr.

The evaporation apparatus as described above is suitably applicable not only to the film formation of an organic EL raw material, but also to the film formation of a lithium (Li) metal having a low vapor pressure. Deposition is carried out by heating Li with a melting point of 179° C. to 200 to 250° C. in the evaporating jig 55 and reducing the pressure to 1×10⁻⁵ Torr in an Ar atmosphere, and then the film formation can be stopped by increasing the pressure of the Ar atmosphere to 10 Torr. Using the evaporating jig 55 of this invention, since the evaporation rate is temporally uniform over a large area, uniform thickness deposition can be carried out on the surface of a large-area substrate and even on the surfaces of different substrates.

### INDUSTRIAL APPLICABILITY

According to this invention, a high-quality organic EL device can be obtained by applying it to organic EL film formation. Further, this invention is applicable not only to the organic EL film formation, but also to film formation for various display devices and so on requiring high quality and long lifetime. In the foregoing examples, the description has been given of the case where the plane shape of the small openings of the evaporating jig is rectangular. However, it is clear that the shape is not limited to a rectangle, but may be a square, a regular triangle, a regular pentagon, a regular hexagon, a regular octagon, other polygons, a rhombus, a circle, an ellipse, a clover shape, a cross shape (with a linear or curved periphery), and other shapes. Further, with respect also to the partition plate, the shape is not limited to the plate shape, but may be another shape such as a corrugated plate shape, a rod shape, or a mesh shape as long as having a structure that can prevent the thermal convection. Further, even with a structure in which each of small openings is, as seen from above, not completely surrounded by a partition along its periphery so that the small openings communicate with each other at their surfaces, no problem arises as long as the structure can prevent the thermal convection. In this case, the communication structure at the bottom can be omitted. Alternatively, even if regions, corresponding to the small openings, communicating with each other are formed by post-like or rod-like members projecting upward from the bottom of the container, no problem arises as long as the structure can prevent the thermal convection.

Further, it is preferable that the evaporating container itself and the partition plate or posts be formed of a material having good heat conduction and their surfaces to be in contact with a molten liquid be formed as surfaces having a low catalytic effect with respect to an organic EL material. The surface with the low catalytic effect is, for example, a passive film of alumina, yttria, or the like, carbon, fluorocarbon, or the like.

The shape of the container is preferably configured such that the evaporation area does not change even when the liquid surface is lowered. For example, the shape with perpendicular inner surfaces is preferable such as a cube, a rectangular parallelepiped, a circular cylinder, or a prism. Likewise, the partition member is also preferably configured such that the evaporation area does not change even when the liquid surface is lowered.

Since the evaporation rate is sensitive to the pressure and the temperature, it is necessary to precisely control the pressure and the temperature so as to be constant. Further, a carrier gas is supplied to the liquid surface so that its temperature and flow rate become constant. For this purpose, it is preferable to provide filters such as slit filters at an inlet to and an outlet from the evaporating container.

1. A film forming apparatus that evaporates a raw material by an evaporation unit and supplies said evaporated raw material onto a substrate, thereby forming a film of a predetermined material on said substrate, said film forming apparatus wherein said evaporation unit comprises a container having an opening and a bottom surface and a partition member disposed in said container and extending in a direction from said opening toward said bottom surface.

2. A film forming apparatus according to claim 1, wherein said partition member is provided so as to continuously or partially cross said opening and is configured so that a space continues at a bottom portion or a side portion of said partition member.

3. A film forming apparatus according to claim 1, wherein said partition member is configured so as to prevent occurrence of thermal convection and to make a liquid surface uniform when said raw material is in a liquid state and is subjected to an evaporation treatment in said container.

4. A film forming apparatus according to claim 1, further comprising supply means for a carrier gas that transports said evaporated raw material onto said substrate, wherein a concentration of said raw material in said carrier gas is constant.

5. A film forming apparatus that evaporates a raw material used for forming a film of a predetermined material and supplies said raw material evaporated onto a substrate, thereby forming the film of the predetermined material on said substrate, said film forming apparatus wherein an evaporation unit for evaporating said raw material comprises a heat resistant container having an opening of a predetermined area at one end thereof and adapted to contain therein said raw material in a liquid state and a dividing unit for dividing said opening of said container into a plurality of partial spaces each having an area smaller than said predetermined area, wherein said dividing unit has a portion that continuously or partially crosses said opening and a portion that causes sad partial spaces to communicate with each other at least one of a bottom portion or said opening of said container.

6. An evaporating jig for use in evaporating a filled raw material, said evaporating jig comprising an evaporating dish having a bottom surface and a side surface standing from said bottom surface and defining an opening and a raw material containing space by said bottom surface and said side surface and a partition member received in said raw material containing space and extending in a direction from said opening toward said bottom surface.

7. An evaporating jig according to claim 6, wherein said partition member is provided so as to continuously or partially cross said opening and is configured so that a space continues at a bottom portion or a side portion of said partition member.

8. An evaporating jig according to claim 6, wherein said partition member is in contact with said bottom surface at least a portion of a bottom portion of said partition member and has a communication hole near said bottom surface.

9. An evaporating jig according to claim 6, wherein said opening has a rectangular shape with a long side and a short side and said raw material containing space has a rectangular parallelepiped shape, wherein said partition member comprises a long-side direction partition piece extending in a long-side direction and a short-side direction partition piece extending in a short-side direction.

10. An evaporating jig for use in evaporating a filled liquid-state raw material, said evaporating jig comprising an evaporating dish having a bottom surface and a side surface standing from said bottom surface and defining an open raw material containing space inside said side surface and a partition plate dividing said raw material containing space into a plurality of partial spaces, wherein said partition plate is retained in said evaporating dish so that said plurality of partial spaces communicate with each other on a bottom surface side of said evaporating dish.

11. An evaporating jig according to claim 10, wherein said evaporating dish defines the raw material containing space having a predetermined length, width, and depth and a rectangular or square opening and said partition plate comprises a partition piece extending in a length direction of said evaporating dish and a partition piece extending in a width direction of said evaporating dish, wherein said partition pieces each have a height smaller than the depth of said raw material containing space.

12. An evaporating jig according to claim 10, wherein said side surface has, at its upper portion, a structure that prevents creeping-up of said liquid-state raw material.

13. An evaporating jig according to claim 10, wherein said partial spaces are configured so as to each have a polygonal shape as seen from above.

14. An evaporating jig for use in evaporating a filled liquid-state raw material, said evaporating jig comprising an evaporating dish having a bottom surface and a side surface standing from said bottom surface and defining an open raw material containing space inside said side surface and a partition plate dividing said raw material containing space into a plurality of partial spaces, wherein said partition plate is configured to be discontinuous in a direction parallel to said bottom surface and is retained in said evaporating dish so that said plurality of partial spaces communicate with each other in said parallel direction.

15. An evaporating jig according to any one of claims 10 to 14 claim 10 or 14, further comprising a heating unit for heating said evaporating dish.

16. An evaporating jig according to claim 15, further comprising means for heating said partition plate.

17. An evaporating jig according to claim 16, wherein each of said heating units includes a heat pipe.

18. An evaporating jig according to claim 10 or 14, further comprising means for supplying a carrier gas to said evaporating dish.

19. An evaporating jig according to claim 18, wherein said carrier gas is supplied through a filter.

20. A measurement method that causes an organic raw material to be contained in the evaporating jig according to any one of claims 6, 10 or 14 and evaporates said organic raw material therein, and transports said evaporated organic raw material using a carrier gas, thereby measuring a concentration of said evaporated organic raw material in said carrier gas.

21. A measurement method including calculating an activation energy of said organic raw material from said concentration measured by the measurement method according to claim 20.

22. A measurement method including deriving, from said activation energy obtained by the measurement method according to claim 21, said measured concentration, and a temperature of said raw material, a constant Ko of a formula (I):
V=(Ko/P)×e^{-Ea/kT}
(where Ko is a constant (%·Torr), P is a pressure (Torr), Ea is an activation energy (eV), k is a Boltzmann constant, and T is an absolute temperature)
representing the concentration V (%) of said evaporated organic raw material in said carrier gas.

23. A measurement method including predicting said organic raw material from a calculation result of the constant Ko obtained by the measurement method according to claim 22.

24. A measurement method according to claim 20, wherein said organic raw material is a raw material of an organic electroluminescence element.

25. A film forming method including causing an organic raw material to be contained in the evaporating jig according to claim 6, and evaporating said organic raw material therein, and transporting said evaporated organic raw material using a carrier gas, thereby depositing a film of said organic raw material on a substrate.

26. A film forming method including causing a liquid-state raw material to be contained in the evaporating jig according to claim 10 or 14, and evaporating said liquid-state raw material therein, and transporting said evaporated raw material using a carrier gas, thereby depositing a film of said raw material on a substrate.

27. A film forming method including causing a liquid-state raw material to be contained in the evaporating jig according to claim 10 or 14, and evaporating said liquid-state raw material at a reduced pressure therein, thereby depositing said evaporated raw material on a lower surface of a substrate disposed over said evaporating jig.
